(19) United States
(12) Patent Application Publication
(10) Pub. No.: US 2020/0248159 A1
(43) Pub. Date: Aug. 6, 2020

(54) POLYPEPTIDES HAVING PROTEASE ACTIVITY AND POLYNUCLEOTIDES ENCODING SAME

(71) Applicant: NOVOZYMES A/S, Bagsvaerd (DK)

(72) Inventors: Louis Patrick Lessard, Wake Forest, NC (US); Kenneth Jensen, Oelsted (DK); Tine Hoff, Holte (DK)

(73) Assignee: NOVOZYMES A/S, Bagsvaerd (DK)

(21) Appl. No.: 16/652,884

(22) PCT Filed: Oct. 3, 2018
(86) PCT No.: PCT/US2018/054212

§ 371 Date: Apr. 1, 2020

Related U.S. Application Data
(60) Provisional application No. 62/567,841, filed on Oct. 4, 2017.

(51) Int. Cl. C12N 9/52; C12N 15/63; C12P 19/14

(52) U.S. Cl. CPC C12N 9/52; C12P 19/14; C12N 15/63

(57) ABSTRACT

The present invention relates to polypeptides having protease activity obtainable from Palaeococcus ferrophilus, in particular proteases selected from the group consisting of: (a) a polypeptide having at least 85%, at least 90%, at least 95%, at least 96%, at least 97%, at least 98%, at least 99%, or 100% sequence identity to the mature polypeptide of SEQ ID NO: 2; (b) a polypeptide encoded by a polynucleotide that hybridizes under very-high stringency conditions with (i) the mature polypeptide coding sequence of SEQ ID NO: 1, (ii) the full-length complement of (i) or (ii); (c) a polypeptide encoded by a polynucleotide having at least 85%, at least 90%, at least 95%, at least 96%, at least 97%, at least 98%, at least 99%, or 100% sequence identity to the mature polypeptide coding sequence of SEQ ID NO: 1; (d) a fragment of the polypeptide of (a), (b), or (c), that has protease activity. and polynucleotides encoding the polypeptides. The invention also relates to nucleic acid constructs, vectors, and host cells comprising the polynucleotides as well as methods of producing and using the polypeptides.

### REFERENCE TO A SEQUENCE LISTING

This application contains a Sequence Listing in computer readable form, which is incorporated herein by reference.

### FIELD OF THE INVENTION

The present invention relates to polypeptides having protease activity, and polynucleotides encoding the polypeptides. The invention also relates to nucleic acid constructs, vectors, and host cells comprising the polynucleotides as well as methods of producing and using the polypeptides.

### BACKGROUND OF THE INVENTION

Fermentation products, such as ethanol, are typically produced by first grinding starch-containing material in a dry-grind or wet-milling process, then degrading the material into fermentable sugars using enzymes and finally converting the sugars directly or indirectly into the desired fermentation product using a fermenting organism. Liquid fermentation products are recovered from the fermented mash (often referred to as “beer mash”), e.g., by distillation, which separate the desired fermentation product from other liquids and/or solids. The remaining faction is referred to as “whole stillage”. The whole stillage is dewatered and separated into a solid and a liquid phase, e.g., by centrifugation. The solid phase is referred to as “wet cake” (or “wet grains”) and the liquid phase (supernatant) is referred to as “thin stillage”. Wet cake and thin stillage contain about 35 and 7% solids, respectively. Dewatered wet cake is dried to provide “Distillers Dried Grains” (DDG) used as nutrient in animal feed. Thin stillage is typically evaporated to provide condensate and syrup or may alternatively be recycled directly to the slurry tank as “backset”. Condensate may either be forwarded to a methanator before being discharged or may be recycled to the slurry tank. The syrup may be blended into DDG or added to the wet cake before drying to produce DDGS (Distillers Dried Grain with Solubles).

WO 2012/088303 (Novozymes) discloses processes for producing fermentation products by liquefying starch-containing material at a pH in the range from 4.5-5.0 at a temperature in the range from 80-90° C. using a combination of alpha-amylase having a T1/2 (min) at pH 4.5, 85° C., 0.12 mM CaCl2) of at least 10 and a protease having a thermostability value of more than 20% determined as Relative Activity at 80° C./70° C.; followed by saccharification and fermentation.

WO 2013/082486 (Novozymes) discloses processes for producing fermentation products by liquefying starch-containing material at a pH in the range between from above 5.0-7.0 at a temperature above the initial gelatinization temperature using an alpha-amylase; a protease having a thermostability value of more than 20% determined as Relative Activity at 80° C./70° C.; and optionally a carbohydrate-source generating enzyme followed by saccharification and fermentation. The process is exemplified using a protease from Pyrococcus furiosus, PfuS.

WO2014/209800 (Novozymes) discloses a process for producing fermentation products by liquefying starch-containing material at a temperature above the initial gelatinization temperature using an alpha-amylase and high dose of the PfuS protease.

An increasing number of ethanol plants extract oil from the thin stillage and/or syrup as a by-product for use in biodiesel production or other biorenewable products. Much of the work in oil recovery/extraction from fermentation product production processes has focused on improving the extractability of the oil from the thin stillage. Effective removal of oil is often accomplished by hexane extraction. However, the utilization of hexane extraction has not seen widespread application due to the high capital investment required. Therefore, other processes that improve oil extraction from fermentation product production processes have been explored.

WO 2011/126897 (Novozymes) discloses processes of recovering oil by converting starch-containing materials into dextrins with alpha-amylase; saccharifying with a carbohydrate source generating enzyme to form sugars; fermenting the sugars using fermenting organism; wherein the fermentation medium comprises a hemicellulase; distilling the fermentation product to form whole stillage; separating the whole stillage into thin stillage and wet cake; and recovering oil from the thin stillage. The fermentation medium may further comprise a protease.

WO 2016/196202 discloses a S8 protease from Thermococcus for use in an ethanol process.

It is an object of the present invention to provide improved processes for increasing the amount of recoverable oil from fermentation product production processes and to provide processes for producing fermentation products, such as ethanol, from starch-containing material that can provide a higher fermentation product yield, or other advantages, compared to a conventional process.

### SUMMARY OF THE INVENTION

The present invention relates to a polypeptide having protease activity, selected from the group consisting of:

(a) a polypeptide having at least 85%, at least 90%, at least 95%, at least 96%, at least 97%, at least 98%, at least 99%, or 100% sequence identity to the mature polypeptide of SEQ ID NO: 2;

(b) a polypeptide encoded by a polynucleotide that hybridizes under very-high stringency conditions with (i) the mature polypeptide coding sequence of SEQ ID NO: 1, (ii) the full-length complement of (i) or (ii);

(c) a polypeptide encoded by a polynucleotide having at least 85%, at least 90%, at least 95%, at least 96%, at least 97%, at least 98%, at least 99%, or 100% sequence identity to the mature polypeptide coding sequence of SEQ ID NO: 1; and

(d) a fragment of the polypeptide of (a), (b), or (c) that has protease activity.

The present invention also relates to polynucleotides encoding the polypeptides of the present invention; nucleic acid constructs; recombinant expression vectors; recombinant host cells comprising the polynucleotides; and methods of producing the polypeptides.

The present invention further relates to a process for liquefying starch-containing material comprising liquefying the starch-containing material at a temperature above the initial gelatinization temperature in the presence of at least an alpha-amylase and a S8A Palaeococcus ferrophilus protease. In a further aspect the invention relates to a process for producing fermentation products from starch-containing material comprising the steps of: a) liquefying the starch-containing material at a temperature above the initial gelatinization temperature in the presence of at least: an alpha-amylase; and a Palaeococcus ferrophilus S8A protease; b) saccharifying using a glucoamylase; c) fermenting using a fermenting organism.

The present invention further relates to a process of recovering oil from a fermentation product production comprising the steps of: a) liquefying the starch-containing material at a temperature above the initial gelatinization temperature in the presence of at least: an alpha-amylase; and a Palaeococcus ferrophilus S8A protease of the invention; b) saccharifying using a glucoamylase; c) fermenting using a fermenting organism; d) recovering the fermentation product to form whole stillage; e) separating the whole stillage into thin stillage and wet cake; f) optionally concentrating the thin stillage into syrup; wherein oil is recovered from the: liquefied starch-containing material after step a) of the process; and/or downstream from fermentation step c) of the process.

The present invention further relates to an enzyme composition comprising a Palaeococcus ferrophilus S8A protease of the invention.

In a still further aspect the invention relates to a use of a Palaeococcus ferrophilus S8A protease in liquefaction of starch-containing material.

### Definitions

S8A Protease: The term “S8A protease” means an S8 protease belonging to subfamily A. Subtilisins, EC 3.4.21.62, are a subgroup in subfamily S8A, however, the present S8A protease from Palaeococcus ferrophilus is a subtilisin-like protease, which has not yet been included in the IUBMB classification system. The S8A protease according to the invention hydrolyses the substrate Suc-Ala-Ala-Pro-Phe-pNA. The release of p-nitroaniline (pNA) results in an increase of absorbance at 405 nm and is proportional to the enzyme activity.

In one aspect, the polypeptides of the present invention have at least 20%, e.g., at least 40%, at least 50%, at least 60%, at least 70%, at least 80%, at least 90%, at least 95%, or at least 100% of the protease activity of the mature polypeptide of SEQ ID NO: 2. In one embodiment protease activity can be determined by the kinetic Suc-AAPF-pNA assay as disclosed in example 2.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign/heterologous (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide having one or more (e.g., several) amino acids absent from the amino and/or carboxyl terminus of a mature polypeptide or domain; wherein the fragment has protease activity. In one aspect, a fragment contains at least 325 amino acid residues (e.g., amino acids 101 to 425 of SEQ ID NO: 2).

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). An isolated substance may be present in a fermentation broth sample; e.g. a host cell may be genetically modified to express the polypeptide of the invention. The fermentation broth from that host cell will comprise the isolated polypeptide.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 101 to 425 of SEQ ID NO: 2. Amino acids 1 to 24 of SEQ ID NO: 2 are a signal peptide. Amino acids 25 to 100 are a pro-peptide.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide. The N-terminal was confirmed by MS-EDMAN data on the purified protease as shown in the examples section.

Mature polypeptide coding sequence: The term “mature polypeptide coding sequence” means a polynucleotide that encodes a mature polypeptide having protease activity. In one aspect, the mature polypeptide coding sequence is nucleotides 1 to 1275 of SEQ ID NO: 1.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

Stringency conditions: The term “very low stringency conditions” means for probes of at least 100 nucleotides in length, prehybridization and hybridization at 42° C. in 5× SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2× SSC, 0.2% SDS at 45° C.

The term “low stringency conditions” means for probes of at least 100 nucleotides in length, prehybridization and hybridization at 42° C. in 5× SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2× SSC, 0.2% SDS at 50° C.

The term “medium stringency conditions” means for probes of at least 100 nucleotides in length, prehybridization and hybridization at 42° C. in 5× SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2× SSC, 0.2% SDS at 55° C.

The term “medium-high stringency conditions” means for probes of at least 100 nucleotides in length, prehybridization and hybridization at 42° C. in 5× SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2× SSC, 0.2% SDS at 60° C.

The term “high stringency conditions” means for probes of at least 100 nucleotides in length, prehybridization and hybridization at 42° C. in 5× SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2× SSC, 0.2% SDS at 65° C.

The term “very high stringency conditions” means for probes of at least 100 nucleotides in length, prehybridization and hybridization at 42° C. in 5× SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2× SSC, 0.2% SDS at 70° C.

Subsequence: The term “subsequence” means a polynucleotide having one or more (e.g., several) nucleotides absent from the 5′ and/or 3′ end of a mature polypeptide coding sequence; wherein the subsequence encodes a fragment having protease activity.

Variant: The term “variant” means a polypeptide having protease activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. In describing variants, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions. For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different alterations. Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants:

“Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

### Polypeptides Having Protease Activity

In an embodiment, the present invention relates to polypeptides having a sequence identity to the mature polypeptide of SEQ ID NO: 2 of at least 85%, at least 90%, 95%, at least 96%, at least 97%, at least 98%, at least 99%, or 100%, which have protease activity. In one aspect, the polypeptides differ by up to 10 amino acids, e.g., 1, 2, 3, 4, 5, 6, 7, 8, 9, or 10, from the mature polypeptide of SEQ ID NO: 2.

In a particular embodiment the invention relates to polypeptides having a sequence identity to the mature polypeptide of SEQ ID NO: 2 of at least 85%, at least 90%, at least 95%, at least 96%, at least 97%, at least 98%, at least 99%, or 100%, and wherein the polypeptide has at least 75% of the protease activity of the mature polypeptide of SEQ ID NO: 2.

In a particular embodiment the invention relates to polypeptides having a sequence identity to the mature polypeptide of SEQ ID NO: 2 of at least 85%, at least 90%, at least 95%, at least 96%, at least 97%, at least 98%, at least 99%, or 100%, and wherein the polypeptide has at least 80% of the protease activity of the mature polypeptide of SEQ ID NO: 2.

In a particular embodiment the invention relates to polypeptides having a sequence identity to the mature polypeptide of SEQ ID NO: 2 of at least 85%, at least 90%, at least 95%, at least 96%, at least 97%, at least 98%, at least 99%, or 100%, and wherein the polypeptide has at least 85% of the protease activity of the mature polypeptide of SEQ ID NO: 2.

In a particular embodiment the invention relates to polypeptides having a sequence identity to the mature polypeptide of SEQ ID NO: 2 of at least 85%, at least 90%, at least 95%, at least 96%, at least 97%, at least 98%, at least 99%, or 100%, and wherein the polypeptide has at least 90% of the protease activity of the mature polypeptide of SEQ ID NO: 2.

In a particular embodiment the invention relates to polypeptides having a sequence identity to the mature polypeptide of SEQ ID NO: 2 of at least 85%, at least 90%, at least 95%, at least 96%, at least 97%, at least 98%, at least 99%, or 100%, and wherein the polypeptide has at least 95% of the protease activity of the mature polypeptide of SEQ ID NO: 2.

In a particular embodiment the invention relates to polypeptides having a sequence identity to the mature polypeptide of SEQ ID NO: 2 of at least 85%, at least 90%, at least 95%, at least 96%, at least 97%, at least 98%, at least 99%, or 100%, and wherein the polypeptide has at least at least 96% of the protease activity of the mature polypeptide of SEQ ID NO: 2.

In a particular embodiment the invention relates to polypeptides having a sequence identity to the mature polypeptide of SEQ ID NO: 2 of at least 85%, at least 90%, at least 95%, at least 96%, at least 97%, at least 98%, at least 99%, or 100%, and wherein the polypeptide has at least at least 97% of the protease activity of the mature polypeptide of SEQ ID NO: 2.

In a particular embodiment the invention relates to polypeptides having a sequence identity to the mature polypeptide of SEQ ID NO: 2 of at least 85%, at least 90%, at least 95%, at least 96%, at least 97%, at least 98%, at least 99%, or 100%, and wherein the polypeptide has at least at least 98% of the protease activity of the mature polypeptide of SEQ ID NO: 2.

In a particular embodiment the invention relates to polypeptides having a sequence identity to the mature polypeptide of SEQ ID NO: 2 of at least 85%, at least 90%, at least 95%, at least 96%, at least 97%, at least 98%, at least 99%, or 100%, and wherein the polypeptide has at least at least 99% of the protease activity of the mature polypeptide of SEQ ID NO: 2.

The polynucleotides of SEQ ID NO: 1, or subsequences thereof, as well as the polypeptides of SEQ ID NO: 2 or a fragments thereof may be used to design nucleic acid probes to identify and clone DNA encoding polypeptides having protease activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having protease activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1 or subsequences thereof, the carrier material is used in a Southern blot.

For purposes of the present invention, hybridization indicates that the polynucleotide hybridizes to a labeled nucleic acid probe corresponding to (i) SEQ ID NO: 1; (ii) the mature polypeptide coding sequence of SEQ ID NO: 1; (iii) the full-length complement thereof; or (iv) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is nucleotides 1 to 1275 of SEQ ID NO: 1. In another aspect, the nucleic acid probe is a polynucleotide that encodes the polypeptide of SEQ ID NO: 2; the mature polypeptide thereof; or a fragment thereof. In another aspect, the nucleic acid probe is SEQ ID NO: 1.

In another embodiment, the present invention relates to a polypeptide having protease activity encoded by a polynucleotide having a sequence identity to the mature polypeptide coding sequence of SEQ ID NO: 1 of at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 2 comprising a substitution, deletion, and/or insertion at one or more (e.g., several) positions. In an embodiment, the number of amino acid substitutions, deletions and/or insertions introduced into the mature polypeptide of SEQ ID NO: 2 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for protease activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Sources of Polypeptides Having Protease Activity

A polypeptide having protease activity of the present invention may be obtained from microorganisms of the genus Palaeococcus.

In another aspect, the polypeptide is a Palaeococcus ferrophilus polypeptide.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to polynucleotides encoding a polypeptide of the present invention, as described herein. In an embodiment, the polynucleotide encoding the polypeptide the present invention has been isolated.

The techniques used to isolate or clone a polynucleotide are known in the art and include isolation from genomic DNA or cDNA, or a combination thereof. The cloning of the polynucleotides from genomic DNA can be effected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Palaeococcus, particularly Palaeococcus ferrophilus, or a related organism and thus, for example, may be an allelic or species variant of the polypeptide encoding region of the polynucleotide.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences. In a particular embodiment, at least one control sequence is heterologous to the polynucleotide encoding a variant of the present invention. Thus, the nucleic acid construct would not be found in nature.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCI B 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

### Expression Vectors

The present invention also relates to recombinant expression vectors comprising a polynucleotide of the present invention, a promoter, and transcriptional and translational stop signals. The polynucleotide and control sequences may be joined together to produce a recombinant expression vector that may include one or more convenient restriction sites to allow for insertion or substitution of the polynucleotide encoding the polypeptide at such sites. In a particular embodiment at least one control sequence is heterologous to the polynucleotide of the present invention. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. In one embodiment the one or more control sequences are heterologous to the polynucleotide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, andUreaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide. In one aspect, the cell is a Palaeococcus ferrophilus cell, in particular DSM13482.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Enzyme Compositions

The present invention also relates to compositions comprising a polypeptide of the present invention.

The compositions may comprise a protease of the present invention as the major enzymatic component, e.g., a mono-component composition. Alternatively, the compositions may comprise multiple enzymatic activities, such as one or more (e.g., several) enzymes selected from the group consisting of alpha-amylase, glucoamylase, beta-amylase, pullulanase.

The compositions may be prepared in accordance with methods known in the art and may be in the form of a liquid or a dry composition. The compositions may be stabilized in accordance with methods known in the art.

Examples are given below of preferred uses of the compositions of the present invention. An enzyme composition of the invention comprises an alpha-amylase and a Palaeococcus ferrophilus S8A protease suitable for use in a liquefaction step in a process of the invention.

In a particular embodiment the invention relates to an enzyme composition comprising:
an alpha-amylase and a Palaeococcus ferrophilus S8A protease, in particular a protease having at least 85%, at least 90%, at least 95%, at least 96%, at least 97%, at least 98%, at least 99%, or 100% sequence identity to the mature polypeptide of SEQ ID NO: 2.

In a preferred embodiment the ratio between alpha-amylase and protease is in the range from 1:1 and 1:50 (micro gram alpha-amylase: micro gram protease), more particularly in the range between 1:3 and 1:40, such as around 1:4 (micro gram alpha-amylase: micro gram protease).

In a preferred embodiment the enzyme composition of the invention comprises a glucoamylase and the ratio between alpha-amylase and glucoamylase in liquefaction is between 1:1 and 1:10, such as around 1:2 (micro gram alpha-amylase: micro gram glucoamylase).

The alpha-amylase is preferably a bacterial acid stable alpha-amylase. Particularly the alpha-amylase is from an Exiguobacterium sp. or a Bacillus sp. such as e.g., Bacillus stearothermophilus or Bacillus licheniformis.

In an embodiment the alpha-amylase is from the genus Bacillus, such as a strain of Bacillus stearothermophilus, in particular a variant of a Bacillus stearothermophilus alpha-amylase, such as the one shown in SEQ ID NO: 3 in WO 99/019467 or SEQ ID NO: 4 herein.

In an embodiment the Bacillus stearothermophilus alpha-amylase or variant thereof is truncated, preferably to have around 491 amino acids, such as from 480-495 amino acids.

In an embodiment the Bacillus stearothermophilus alpha-amylase has a deletion at two positions within the range from positions 179 to 182, such as positions I181+G182, R179+G180, G180+I181, R179+I181, or G180+G182, preferably I181+G182, and optionally a N193F substitution, (using SEQ ID NO: 4 for numbering).

In an embodiment the Bacillus stearothermophilus alpha-amylase has a substitution at position S242, preferably S242Q substitution.

In an embodiment the Bacillus stearothermophilus alpha-amylase has a substitution at position E188, preferably E188P substitution.

In an embodiment the alpha-amylase is selected from the group of Bacillus stearothermophilus alpha-amylase variants with the following mutations in addition to a double deletion in the region from position 179 to 182, particularly I181*+G182* and optionally N193F:

V59A + Q89R + G112D + E129V + K177L + R179E + K220P + N224L + Q254S;
V59A + Q89R + E129V + K177L + R179E + H208Y + K220P + N224L + Q254S;
V59A + Q89R + E129V + K177L + R179E + K220P + N224L + Q254S + D269E + D281N;
V59A + Q89R + E129V + K177L + R179E + K220P + N224L + Q254S + I270L;
V59A + Q89R + E129V + K177L + R179E + K220P + N224L + Q254S + H274K;
V59A + Q89R + E129V + K177L + R179E + K220P + N224L + Q254S + Y276F;
V59A + E129V + R157Y + K177L + R179E + K220P + N224L + S242Q + Q254S;
V59A + E129V + K177L + R179E + H208Y + K220P + N224L + S242Q + Q254S;
59A + E129V + K177L + R179E + K220P + N224L + S242Q + Q254S;
V59A + E129V + K177L + R179E + K220P + N224L + S242Q + Q254S + H274K;
V59A + E129V + K177L + R179E + K220P + N224L + S242Q + Q254S + Y276F;
V59A + E129V + K177L + R179E + K220P + N224L + S242Q + Q254S + D281N;
V59A + E129V + K177L + R179E + K220P + N224L + S242Q + Q254S + M284T;
V59A + E129V + K177L + R179E + K220P + N224L + S242Q + Q254S + G416V;
V59A + E129V + K177L + R179E + K220P + N224L + Q254S;
V59A + E129V + K177L + R179E + K220P + N224L + Q254S + M284T;
A91L + M96I + E129V + K177L + R179E + K220P + N224L+ S242Q + Q254S;
E129V + K177L + R179E;
E129V + K177L + R179E + K220P + N224L + S242Q + Q254S;
E129V + K177L + R179E + K220P + N224L + S242Q + Q254S + Y276F + L427M;
E129V + K177L + R179E + K220P + N224L + S242Q + Q254S + M284T;
E129V + K177L + R179E + K220P + N224L + S242Q + Q254S + N376* + I377*;
E129V + K177L + R179E + K220P + N224L + Q254S;
E129V + K177L + R179E + K220P + N224L + Q254S + M284T;
E129V + K177L + R179E + S242Q;
E129V + K177L + R179V + K220P + N224L + S242Q + Q254S;
K220P + N224L + S242Q + Q254S;
M284V;
V59A Q89R + E129V + K177L+ R179E + Q254S + M284V.

In an embodiment the alpha-amylase is selected from the group of Bacillus stearothermophilus alpha-amylase variants with the following mutations:
I181*+G182*+N193F+E129V+K177L+R179E;
I181*+G182*+N193F+V59A+Q89R+E129V+K177L+R179E+H208Y+K220P+N224L+Q254S;
I181*+G182*+N193F+V59A Q89R+E129V+K177L+R179E+Q254S+M284V; and
I181*+G182*+N193F+E129V+K177L+R179E+K220P+N224L+S242Q+Q254S (using SEQ ID NO: 4 for numbering).

In an embodiment the alpha-amylase variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 4.

In a preferred embodiment the enzyme composition of the invention, comprises a Palaeococcus ferrophilus S8A protease having at least 80%, such as at least 85%, such as at least 90%, such as at least 95%, such as at least 96%, such as at least 97%, such as at least 98%, such as at least 99%, or at least 100% identity to amino acids 101 to 425 of SEQ ID NO: 2.

In an embodiment the enzyme composition further comprises a glucoamylase.

In an embodiment the glucoamylase is derived from a strain of the genus Penicillium, especially a strain of Penicillium oxalicum disclosed as SEQ ID NO: 2 in WO 2011/127802.

In an embodiment the glucoamylase has at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99% or 100% identity to the mature polypeptide of SEQ ID NO: 2 in WO 2011/127802 or SEQ ID NO: 11 herein.

In an embodiment the glucoamylase is a variant of the Penicillium oxalicum glucoamylase disclosed as SEQ ID NO: 2 in WO 2011/127802 herein having a K79V substitution such as a variant disclosed in WO 2013/053801.

In an embodiment the glucoamylase is the Penicillium oxalicum glucoamylase having a K79V substitution and further one of the following substitutions:
P11F+T65A+Q327F
P2N+P4S+P11F+T65A+Q327F.

In an embodiment the composition further comprises a pullulanase.

In an embodiment the composition of the invention comprises a Bacillus stearothermophilus alpha-amylase and a Palaeococcus ferrophilus S8A protease; In one embodiment the ratio between alpha-amylase and protease is in the range from 1:1 and 1:50 (micro gram alpha-amylase: micro gram protease).

In an embodiment the ratio between alpha-amylase and protease is in the range between 1:3 and 1:40, such as around 1:4 (micro gram alpha-amylase : micro gram protease).

In an embodiment the ratio between alpha-amylase and glucoamylase is between 1:1 and 1:10, such as around 1:2 (micro gram alpha-amylase : micro gram glucoamylase).

### Processes of the Invention

The present invention relates to processes of recovering oil from a fermentation product production process and well as processes for producing fermentation products from starch-containing material.

The inventors have found that an increased in ethanol yields can be obtained in a processes for producing fermentation products from starch-containing material when combining an alpha-amylase and a protease from Palaeococcus ferrophilus in liquefaction. Thus in one aspect the invention relates to a process for liquefying starch-containing material comprising liquefying the starch-containing material at a temperature above the initial gelatinization temperature in the presence of at least an alpha-amylase and a S8A Palaeococcus ferrophilus protease of the invention, particularly a protease having at least 85%, at least 90%, at least 95%, at least 96%, at least 97%, at least 98%, at least 99%, or 100% sequence identity to the mature polypeptide of SEQ ID NO: 2.

It was also found that an ethanol process of the invention can be run efficiently with reduced or without adding a nitrogen source, such as urea, in SSF.

### Process of Producing a Fermentation Product of the Invention

In a particular aspect the invention relates to processes for producing fermentation products from starch-containing material comprising the steps of:
a) liquefying the starch-containing material at a temperature above the initial gelatinization temperature in the presence of at least:
an alpha-amylase; and
a S8A protease from Palaeococcus ferrophilus;
b) saccharifying using a glucoamylase;
c) fermenting using a fermenting organism.
In an embodiment the fermentation product is recovered after fermentation. In a preferred embodiment the fermentation product is recovered after fermentation, such as by distillation. In an embodiment the fermentation product is an alcohol, preferably ethanol, especially fuel ethanol, potable ethanol and/or industrial ethanol.

### Processes of Recovering/Extracting Oil of the Invention

In another particular aspect the invention relates to processes of recovering oil from a fermentation product production process comprising the steps of:

a) liquefying starch-containing material at a temperature above the initial gelatinization temperature in the presence of at least:
an alpha-amylase; and
a S8A protease from Palaeococcus ferrophilus;

b) saccharifying using a glucoamylase;

c) fermenting using a fermenting organism.

d) recovering the fermentation product to form whole stillage;

e) separating the whole stillage into thin stillage and wet cake;

f) optionally concentrating the thin stillage into syrup;

wherein oil is recovered from the:
liquefied starch-containing material after step a); and/or
downstream from fermentation step c).

In an embodiment the oil is recovered/extracted during and/or after liquefying the starch-containing material. In an embodiment the oil is recovered from the whole stillage. In an embodiment the oil is recovered from the thin stillage. In an embodiment the oil is recovered from the syrup.

In a preferred embodiment of the processes of the invention saccharification and fermentation is performed simultaneously.

In a preferred embodiment no nitrogen-compound, such as urea, is present and/or added in steps a)-c), such as during saccharification step b) or fermentation step c) or simultaneous saccharification and fermentation (SSF).

In an embodiment 10-1,000 ppm, such as 50-800 ppm, such as 100-600 ppm, such as 200-500 ppm nitrogen-compound, preferably urea, is present and/or added in steps a)-c), such as during saccharification step b) or fermentation step c) or simultaneous saccharification and fermentation (SSF).

In an embodiment between 0.5-100 micro gram Palaeococcus ferrophilus S8A protease per gram DS (dry solids) DS is present and/or added in liquefaction step a). In an embodiment between 1-50 micro gram Palaeococcus ferrophilus S8A protease per gram DS (dry solids) DS is present and/or added in liquefaction step a). In an embodiment between 2-40 micro gram Palaeococcus ferrophilus S8A protease per gram DS is present and/or added in liquefaction step a). In an embodiment between 4-25 micro gram Palaeococcus ferrophilus S8A protease per gram DS is present and/or added in liquefaction step a). In an embodiment between 5-20 micro gram Palaeococcus ferrophilus S8A protease per gram DS is present and/or added in liquefaction step a). In an embodiment around or more than 1 micro gram Palaeococcus ferrophilus S8A protease per gram DS is present and/or added in liquefaction step a). In an embodiment around or more than 2 micro gram Palaeococcus ferrophilus S8A protease per gram DS is present and/or added in liquefaction step a). In an embodiment around or more than 5 micro gram Palaeococcus ferrophilus S8A protease per gram DS is present and/or added in liquefaction step a).

### Alpha-Amylases Present and/or Added in Liquefaction

The alpha-amylase added during liquefaction step a) in a process of the invention (i.e., oil recovery process and fermentation product production process) may be any alpha-amylase.

Preferred are bacterial alpha-amylases, which typically are stable at a temperature used in liquefaction.

In an embodiment the alpha-amylase is from a strain of the genus Exiguobacterium or Bacillus.

In a preferred embodiment the alpha-amylase is from a strain of Bacillus stearothermophilus, such as the sequence shown in SEQ ID NO: 3 in W099/019467 or in SEQ ID NO: 4 herein. In an embodiment the alpha-amylase is the Bacillus stearothermophilus alpha-amylase shown in SEQ ID NO: 4 herein, such as one having at least 80%, such as at least 85%, such as at least 90%, such as at least 95%, such as at least 96%, such as at least 97%, such as at least 98%, such as at least 99% identity to SEQ ID NO: 4 herein.

In an embodiment the Bacillus stearothermophilus alpha-amylase or variant thereof is truncated, preferably at the C-terminal, preferably truncated to have around 491 amino acids, such as from 480-495 amino acids.

In an embodiment the Bacillus stearothermophilus alpha-amylase has a deletion at two positions within the range from positions 179 to 182, such as positions I181+G182, R179+G180, G180+I181, R179+I181, or G180+G182, preferably I181+G182, and optionally a N193F substitution, (using SEQ ID NO: 4 for numbering).

In an embodiment the Bacillus stearothermophilus alpha-amylase has a substitution at position S242, preferably S242Q substitution.

In an embodiment the Bacillus stearothermophilus alpha-amylase has a substitution at position E188, preferably E188P substitution.

In an embodiment the alpha-amylase is selected from the group of Bacillus stearothermophilus alpha-amylase variants with the following mutations in addition to a double deletion in the region from position 179 to 182, particularly I181*+G182*, and optionally N193F:

V59A + Q89R + G112D + E129V + K177L + R179E + K220P + N224L + Q254S;
V59A + Q89R + E129V + K177L + R179E + H208Y + K220P + N224L + Q254S;
V59A + Q89R + E129V + K177L + R179E + K220P + N224L + Q254S + D269E + D281N;
V59A + Q89R + E129V + K177L + R179E + K220P + N224L + Q254S + I270L;
V59A + Q89R + E129V + K177L + R179E + K220P + N224L + Q254S + H274K;
V59A + Q89R + E129V + K177L + R179E + K220P + N224L + Q254S + Y276F;
V59A + E129V + R157Y + K177L + R179E + K220P + N224L + S242Q + Q254S;
V59A + E129V + K177L + R179E + H208Y + K220P + N224L + S242Q + Q254S;
59A + E129V + K177L + R179E + K220P + N224L + S242Q + Q254S;
V59A + E129V + K177L + R179E + K220P + N224L + S242Q + Q254S + H274K;
V59A + E129V + K177L + R179E + K220P + N224L + S242Q + Q254S + Y276F;
V59A + E129V + K177L + R179E + K220P + N224L + S242Q + Q254S + D281N;
V59A + E129V + K177L + R179E + K220P + N224L + S242Q + Q254S + M284T;
V59A + E129V + K177L + R179E + K220P + N224L + S242Q + Q254S + G416V;
V59A + E129V + K177L + R179E + K220P + N224L + Q254S;
V59A + E129V + K177L + R179E + K220P + N224L + Q254S + M284T;
A91L + M96I + E129V + K177L + R179E + K220P + N224L + S242Q + Q254S;
E129V + K177L + R179E;
E129V + K177L + R179E + K220P + N224L + S242Q + Q254S;
E129V + K177L + R179E + K220P + N224L + S242Q + Q254S + Y276F + L427M;
E129V + K177L + R179E + K220P + N224L + S242Q + Q254S + M284T;
El 29V + K177L + R179E + K220P + N224L + S242Q + Q254S + N376* + I377*;
E129V + K177L + R179E + K220P + N224L + Q254S;
E129V + K177L + R179E + K220P + N224L + Q254S + M284T;
E129V + K177L + R179E + S242Q;
E129V + K177L + R179V + K220P + N224L + S242Q + Q254S;
K220P + N224L + S242Q + Q254S;
M284V;
V59A Q89R + E129V + K177L + R179E + Q254S + M284V.

In a preferred embodiment the alpha-amylase is selected from the group of Bacillus stearothermophilus alpha-amylase variants:
I181*+G182*+N193F+E129V+K177L+R179E;
I181*+G182*+N193F+V59A+Q89R+E129V+K177L+R179E+H208Y+K220P+N224L+Q254S;
I181*+G182*+N193F+V59A Q89R+E129V+K177L+R179E+Q254S+M284V; and
I181*+G182*+N193F+E129V+K177L+R179E+K220P+N224L+S242Q+Q254S (using SEQ ID NO: 4 for numbering).

According to the invention the alpha-amylase variant has at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 4 herein.

The alpha-amylase may according to the invention be present and/or added in a concentration of 0.1-100 micro gram per gram DS, such as 0.5-50 micro gram per gram DS, such as 1-25 micro gram per gram DS, such as 1-10 micro gram per gram DS, such as 2-5 micro gram per gram DS.

In an embodiment from 1-50 micro gram, particularly from 2-40 micro gram, particularly 4-25 micro gram, particularly 5-20 micro gram Palaeococcus ferrophilus S8A protease per gram DS are present and/or added in liquefaction and 1-10 micro gram Bacillus stearothermophilus alpha-amylase are present and/or added in liquefaction.

In an embodiment the Palaeococcus ferrophilus protease is selected from:

a) a polypeptide comprising or consisting of amino acids 101 to 425 of SEQ ID NO: 2;

b) a polypeptide having at least 80%, at least 85, at least 90%, at least 91%, at least 92%, at least 93%, at least 94%, at least 95%, at least 96%, at least 97%, at least 98%, at least 99%, or 100% sequence identity to amino acids 101 to 425 of SEQ ID NO: 2.

### Glucoamylase Present and/or Added in Liquefaction

In an embodiment a glucoamylase is present and/or added in liquefaction step a) in a process of the invention (i.e., oil recovery process and fermentation product production process).

In a preferred embodiment the glucoamylase present and/or added in liquefaction step a) is derived from a strain of the genus Penicillium, especially a strain of Penicillium oxalicum disclosed as SEQ ID NO: 2 in WO 2011/127802 or SEQ ID NO: 11 herein.

In an embodiment the glucoamylase has at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99% or 100% identity to the mature polypeptide shown in SEQ ID NO: 2 in WO 2011/127802, or SEQ ID NO: 11 herein.

In a preferred embodiment the glucoamylase is a variant of the Penicillium oxalicum glucoamylase shown in SEQ ID NO: 2 in WO 2011/127802 having a K79V substitution, such as a variant disclosed in WO 2013/053801.

In a preferred embodiment the glucoamylase present and/or added in liquefaction is the Penicillium oxalicum glucoamylase having a K79V substitution and preferably further one of the following substitutions:
P11F+T65A+Q327F;
P2N+P4S+P11F+T65A+Q327F.

In an embodiment the glucoamylase variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 2 in

WO 2011/127802 or SEQ ID NO: 11 herein.

The glucoamylase may be added in amounts from 0.1-100 micro grams EP/g, such as 0.5-50 micro grams EP/g, such as 1-25 micrograms EP/g, such as 2-12 micrograms EP/g DS.

### Glucoamylase Present and/or Added in Saccharification and/or Fermentation

A glucoamylase is present and/or added in saccharification and/or fermentation, preferably simultaneous saccharification and fermentation (SSF), in a process of the invention (i.e., oil recovery process and fermentation product production process).

In an embodiment the glucoamylase present and/or added in saccharification and/or fermentation is of fungal origin, preferably from a stain of Aspergillus, preferably A. niger, A. awamori, or A. oryzae; or a strain of Trichoderma, preferably T. reesei; or a strain of Talaromyces, preferably T. emersonii or a strain of Trametes, preferably T. cingulata, or a strain of Pycnoporus, or a strain of Gloeophyllum, such as G. sepiarium or G. trabeum, or a strain of the Nigrofomes.

In an embodiment the glucoamylase is derived from Talaromyces, such as a strain of Talaromyces emersonii, such as the one shown in SEQ ID NO: 5 herein,

In an embodiment the glucoamylase is selected from the group consisting of:

(i) a glucoamylase comprising the polypeptide of SEQ ID NO: 5 herein;

(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 5 herein.

In an embodiment the glucoamylase is derived from a strain of the genus Pycnoporus, in particular a strain of Pycnoporus sanguineus described in WO 2011/066576 (SEQ ID NOs 2, 4 or 6), such as the one shown as SEQ ID NO: 4 in WO 2011/066576.

In an embodiment the glucoamylase is derived from a strain of the genus Gloeophyllum, such as a strain of Gloeophyllum sepiarium or Gloeophyllum trabeum, in particular a strain of Gloeophyllum as described in WO 2011/068803 (SEQ ID NO: 2, 4, 6, 8, 10, 12, 14 or 16). In a preferred embodiment the glucoamylase is the Gloeophyllum sepiarium shown in SEQ ID NO: 2 in WO 2011/068803 or SEQ ID NO: 6 herein.

In a preferred embodiment the glucoamylase is derived from Gloeophyllum sepiarium, such as the one shown in SEQ ID NO: 6 herein. In an embodiment the glucoamylase is selected from the group consisting of:

(i) a glucoamylase comprising the polypeptide of SEQ ID NO: 6 herein;

(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 6 herein.

In another embodiment the glucoamylase is derived from Gloeophyllum trabeum such as the one shown in SEQ ID NO: 7 herein. In an embodiment the glucoamylase is selected from the group consisting of:

(i) a glucoamylase comprising the polypeptide of SEQ ID NO: 7 herein;

(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 7 herein.

In an embodiment the glucoamylase is derived from a strain of the genus Nigrofomes, in particular a strain of Nigrofomes sp. disclosed in WO 2012/064351.

Glucoamylases may in an embodiment be added to the saccharification and/or fermentation in an amount of 0.0001-20 AGU/g DS, preferably 0.001-10 AGU/g DS, especially between 0.01-5 AGU/g DS, such as 0.1-2 AGU/g DS.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U, SPIRIZYME™ ULTRA, SPIRIZYME™ EXCEL and AMG™ E (from Novozymes A/S); OPTIDEX™ 300, GC480, GC417 (from DuPont.); AMIGASE™ and AMIGASE™ PLUS (from DSM); G-ZYME™ G900, G-ZYME™ and G990 ZR (from DuPont).

According to a preferred embodiment of the invention the glucoamylase is present and/or added in saccharification and/or fermentation in combination with an alpha-amylase. Examples of suitable alpha-amylase are described below.

### Alpha-Amylase Present and/or Added in Saccharification and/or Fermentation

In an embodiment an alpha-amylase is present and/or added in saccharification and/or fermentation in a process of the invention. In a preferred embodiment the alpha-amylase is of fungal or bacterial origin. In a preferred embodiment the alpha-amylase is a fungal acid stable alpha-amylase. A fungal acid stable alpha-amylase is an alpha-amylase that has activity in the pH range of 3.0 to 7.0 and preferably in the pH range from 3.5 to 6.5, including activity at a pH of about 4.0, 4.5, 5.0, 5.5, and 6.0.

In a preferred embodiment the alpha-amylase present and/or added in saccharification and/or fermentation is derived from a strain of the genus Rhizomucor, preferably a strain the Rhizomucor pusillus, such as one shown in SEQ ID NO: 3 in WO 2013/006756, such as a Rhizomucor pusillus alpha-amylase hybrid having an Aspergillus niger linker and starch-bonding domain, such as the one shown in SEQ ID NO: 8 herein, or a variant thereof.

In an embodiment the alpha-amylase present and/or added in saccharification and/or fermentation is selected from the group consisting of:

(i) an alpha-amylase comprising the polypeptide of SEQ ID NO: 8 herein;

(ii) an alpha-amylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 8 herein.

In a preferred embodiment the alpha-amylase is a variant of the alpha-amylase shown in SEQ ID NO: 8 having at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C (using SEQ ID NO: 8 for numbering).

In an embodiment the alpha-amylase is derived from a Rhizomucor pusillus with an Aspergillus niger glucoamylase linker and starch-binding domain (SBD), preferably disclosed as SEQ ID NO: 8 herein, preferably having one or more of the following substitutions: G128D, D143N, preferably G128D+D143N (using SEQ ID NO: 8 for numbering).

In an embodiment the alpha-amylase variant present and/or added in saccharification and/or fermentation has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 8 herein.

In a preferred embodiment the ratio between glucoamylase and alpha-amylase present and/or added during saccharification and/or fermentation may preferably be in the range from 500:1 to 1:1, such as from 250:1 to 1:1, such as from 100:1 to 1: 1, such as from 100:2 to 100:50, such as from 100:3 to 100:70.

### Pullulanase Present and/or Added in Liquefaction and/or Saccharification and/or Fermentation

A pullulanase may be present and/or added during liquefaction step a) and/or saccharification step b) or fermentation step c) or simultaneous saccharification and fermentation.

Pullulanases (E.C. 3.2.1.41, pullulan 6-glucano-hydrolase), are debranching enzymes characterized by their ability to hydrolyze the alpha-1,6-glycosidic bonds in, for example, amylopectin and pullulan.

Contemplated pullulanases according to the present invention include the pullulanases from Bacillus amyloderamificans disclosed in U.S. Pat. No. 4,560,651 (hereby incorporated by reference), the pullulanase disclosed as SEQ ID NO: 2 in WO 01/51620 (hereby incorporated by reference), the Bacillus deramificans disclosed as SEQ ID NO: 4 in WO 01/151620 (hereby incorporated by reference), and the pullulanase from Bacillus acidopullulyticus disclosed as SEQ ID NO: 6 in WO 01/51620 and also described in FEMS Mic. Let. (1994) 115, 97-106.

The pullulanase may according to the invention be added in an effective amount which include the preferred amount of about 0.0001-10 mg enzyme protein per gram DS, preferably 0.0001-0.10 mg enzyme protein per gram DS, more preferably 0.0001-0.010 mg enzyme protein per gram DS. Pullulanase activity may be determined as NPUN. An Assay for determination of NPUN is described in the “Materials & Methods”-section below.

Suitable commercially available pullulanase products include PROMOZYME D, PROMOZYME™ D2 (Novozymes A/S, Denmark), OPTIMAX L-300 (Genencor Int., USA), and AMANO 8 (Amano, Japan).

### Further Aspects of Processes of The Invention

Prior to liquefaction step a), processes of the invention, including processes of extracting/recovering oil and processes for producing fermentation products, may comprise the steps of:
i) reducing the particle size of the starch-containing material, preferably by dry milling;
ii) forming a slurry comprising the starch-containing material and water.

In an embodiment at least 50%, preferably at least 70%, more preferably at least 80%, especially at least 90% of the starch-containing material fit through a sieve with # 6 screen.

In an embodiment the pH during liquefaction is between above 4.5-6.5, such as 4.5-5.0, such as around 4.8, or a pH between 5.0-6.2, such as 5.0-6.0, such as between 5.0-5.5, such as around 5.2, such as around 5.4, such as around 5.6, such as around 5.8.

In an embodiment the temperature during liquefaction is above the initial gelatinization temperature, preferably in the range from 70-100° C., such as between 75-95° C., such as between 75-90° C., preferably between 80-90° C., especially around 85° C.

In an embodiment a jet-cooking step is carried out before liquefaction in step a). In an embodiment the jet-cooking is carried out at a temperature between 110-145° C., preferably 120-140° C., such as 125-135° C., preferably around 130° C. for about 1-15 minutes, preferably for about 3-10 minutes, especially around about 5 minutes.

In a preferred embodiment saccharification and fermentation is carried out sequentially or simultaneously.

In an embodiment saccharification is carried out at a temperature from 20-75° C., preferably from 40-70° C., such as around 60° C., and at a pH between 4 and 5.

In an embodiment fermentation or simultaneous saccharification and fermentation (SSF) is carried out carried out at a temperature from 25° C. to 40° C., such as from 28° C. to 35° C., such as from 30° C. to 34° C., preferably around about 32° C. In an embodiment fermentation is ongoing for 6 to 120 hours, in particular 24 to 96 hours.

In a preferred embodiment the fermentation product is recovered after fermentation, such as by distillation.

In an embodiment the fermentation product is an alcohol, preferably ethanol, especially fuel ethanol, potable ethanol and/or industrial ethanol.

In an embodiment the starch-containing starting material is whole grains. In an embodiment the starch-containing material is selected from the group of corn, wheat, barley, rye, milo, sago, cassava, manioc, tapioca, sorghum, rice, and potatoes.

In an embodiment the fermenting organism is yeast, preferably a strain of Saccharomyces, especially a strain of Saccharomyces cerevisae.

In an embodiment the temperature in step (a) is above the initial gelatinization temperature, such as at a temperature between 80-90° C., such as around 85° C.

In an embodiment a process of the invention further comprises a pre-saccharification step, before saccharification step b), carried out for 40-90 minutes at a temperature between 30-65° C. In an embodiment saccharification is carried out at a temperature from 20-75° C., preferably from 40-70° C., such as around 60° C., and at a pH between 4 and 5. In an embodiment fermentation step c) or simultaneous saccharification and fermentation (SSF) (i.e., steps b) and c)) are carried out carried out at a temperature from 25° C. to 40° C., such as from 28° C. to 35° C., such as from 30° C. to 34° C., preferably around about 32° C. In an embodiment the fermentation step c) or simultaneous saccharification and fermentation (SSF) (i.e., steps b) and c)) are ongoing for 6 to 120 hours, in particular 24 to 96 hours.

In an embodiment separation in step e) is carried out by centrifugation, preferably a decanter centrifuge, filtration, preferably using a filter press, a screw press, a plate-and-frame press, a gravity thickener or decker.

In an embodiment the fermentation product is recovered by distillation.

### Fermentation Medium

The environment in which fermentation is carried out is often referred to as the “fermentation media” or “fermentation medium”. The fermentation medium includes the fermentation substrate, that is, the carbohydrate source that is metabolized by the fermenting organism. According to the invention the fermentation medium may comprise nutrients and growth stimulator(s) for the fermenting organism(s). Nutrient and growth stimulators are widely used in the art of fermentation and include nitrogen sources, such as ammonia; urea, vitamins and minerals, or combinations thereof.

### Fermenting Organisms

The term “fermenting organism” refers to any organism, including bacterial and fungal organisms, especially yeast, suitable for use in a fermentation process and capable of producing the desired fermentation product. Especially suitable fermenting organisms are able to ferment, i.e., convert, sugars, such as glucose or maltose, directly or indirectly into the desired fermentation product, such as ethanol. Examples of fermenting organisms include fungal organisms, such as yeast. Preferred yeast includes strains of Saccharomyces spp., in particular, Saccharomyces cerevisiae.

Suitable concentrations of the viable fermenting organism during fermentation, such as SSF, are well known in the art or can easily be determined by the skilled person in the art. In one embodiment the fermenting organism, such as ethanol fermenting yeast, (e.g., Saccharomyces cerevisiae) is added to the fermentation medium so that the viable fermenting organism, such as yeast, count per mL of fermentation medium is in the range from 10⁵ to 10¹², preferably from 10⁷ to 10¹⁰, especially about 5×10⁷.

Examples of commercially available yeast includes, e.g., RED STAR™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, Wisc., USA), BIOFERM AFT and XR (available from NABC—North American Bioproducts Corporation, Ga., USA), GERT STRAND (available from Gert Strand AB, Sweden), and FERMIOL (available from DSM Specialties).

### Starch-Containing Materials

Any suitable starch-containing material may be used according to the present invention. The starting material is generally selected based on the desired fermentation product. Examples of starch-containing materials, suitable for use in a process of the invention, include whole grains, corn, wheat, barley, rye, milo, sago, cassava, tapioca, sorghum, rice, peas, beans, or sweet potatoes, or mixtures thereof or starches derived therefrom, or cereals. Contemplated are also waxy and non-waxy types of corn and barley. In a preferred embodiment the starch-containing material, used for ethanol production according to the invention, is corn or wheat.

### Fermentation Products

The term “fermentation product” means a product produced by a process including a fermentation step using a fermenting organism. Fermentation products contemplated according to the invention include alcohols (e.g., ethanol, methanol, butanol; polyols such as glycerol, sorbitol and inositol); organic acids (e.g., citric acid, acetic acid, itaconic acid, lactic acid, succinic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferably processes of the invention are used for producing an alcohol, such as ethanol. The fermentation product, such as ethanol, obtained according to the invention, may be used as fuel, which is typically blended with gasoline. However, in the case of ethanol it may also be used as potable ethanol.

### Recovery of Fermentation Products

Subsequent to fermentation, or SSF, the fermentation product may be separated from the fermentation medium. The slurry may be distilled to extract the desired fermentation product (e.g., ethanol). Alternatively the desired fermentation product may be extracted from the fermentation medium by micro or membrane filtration techniques. The fermentation product may also be recovered by stripping or other method well known in the art.

### Recovery of Oil

According to the invention oil is recovered during and/or after liquefying, from the whole stillage, from the thin stillage or from the syrup. Oil may be recovered by extraction. In one embodiment oil is recovered by hexane extraction. Other oil recovery technologies well-known in the art may also be used.

The invention is further defined in the following numbered embodiments:

1. A polypeptide having protease activity, selected from the group consisting of:
(a) a polypeptide having at least 85%, at least 90%, at least 95%, at least 96%, at least 97%, at least 98%, at least 99%, or 100% sequence identity to the mature polypeptide of SEQ ID NO: 2;
(b) a polypeptide encoded by a polynucleotide that hybridizes under very-high stringency conditions with (i) the mature polypeptide coding sequence of SEQ ID NO: 1, (ii) the full-length complement of (i) or (ii);
(c) a polypeptide encoded by a polynucleotide having at least 85%, at least 90%, at least 95%, at least 96%, at least 97%, at least 98%, at least 99%, or 100% sequence identity to the mature polypeptide coding sequence of SEQ ID NO: 1; and
(d) a fragment of the polypeptide of (a), (b), or (c) that has protease activity.
2. The polypeptide of embodiment 1, having at least 85%, at least 90%, at least 95%, at least 96%, at least 97%, at least 98%, at least 99% or 100% sequence identity to the mature polypeptide of SEQ ID NO: 2.
3. The polypeptide of embodiment 1 or 2, which is encoded by a polynucleotide that hybridizes under very-high stringency conditions with (i) the mature polypeptide coding sequence of SEQ ID NO: 1, or (ii) the full-length complement of (i).
4. The polypeptide of any of embodiments 1-3, which is encoded by a polynucleotide having at least 80%, at least 85%, at least 90%, at least 95%, at least 96%, at least 97%, at least 98%, at least 99% or 100% sequence identity to the mature polypeptide coding sequence of SEQ ID NO: 1.
5. The polypeptide of any of embodiments 1-4, comprising or consisting of SEQ ID NO: 2 or the mature polypeptide of SEQ ID NO: 2.
6. The polypeptide of embodiment 5, wherein the mature polypeptide is amino acids 101 to 425 of SEQ ID NO: 2.
7. The polypeptide of any of embodiments 1-6, which is a variant of the mature polypeptide of SEQ ID NO: 2 comprising a substitution, deletion, and/or insertion at one or more (several) positions.
8. The polypeptide of embodiment 1, which is a fragment of SEQ ID NO: 2, wherein the fragment has protease activity.
9. A polynucleotide encoding the polypeptide of any of embodiments 1-8.
10. A nucleic acid construct or recombinant expression vector comprising the polynucleotide of embodiment 9 operably linked to one or more heterologous control sequences that direct the production of the polypeptide in an expression host.
11. A recombinant host cell comprising the polynucleotide of embodiment 9 operably linked to one or more heterologous control sequences that direct the production of the polypeptide.
12. A composition comprising the polypeptide of any of embodiments 1-8.
13. A method of producing the polypeptide of any of embodiments 1-8, comprising:
(a) cultivating a cell, which in its wild-type form produces the polypeptide, under conditions conducive for production of the polypeptide and
(b) optionally recovering the polypeptide.
14. A method of producing a polypeptide having protease activity, comprising:
(a) cultivating the host cell of embodiment 11 under conditions conducive for production of the polypeptide; and
(b) optionally recovering the polypeptide.
15. A process for liquefying starch-containing material comprising liquefying the starch-containing material at a temperature above the initial gelatinization temperature in the presence of at least an alpha-amylase and a S8A Palaeococcus ferrophilus protease according to any of embodiments 1-8.
16. A process for producing fermentation products from starch-containing material comprising the steps of:

a) liquefying the starch-containing material at a temperature above the initial gelatinization temperature in the presence of at least:
an alpha-amylase; and
a S8A Palaeococcus ferrophilus protease;

b) saccharifying using a glucoamylase;

c) fermenting using a fermenting organism.

17. A process of recovering oil from a process as disclosed in embodiment 16 further comprising the steps of:

d) recovering the fermentation product to form whole stillage;

e) separating the whole stillage into thin stillage and wet cake;

f) optionally concentrating the thin stillage into syrup;

wherein oil is recovered from the:
liquefied starch-containing material after step a) of the process as disclosed in embodiment 16; and/or
downstream from fermentation step c) of the process as disclosed in embodiment 16.
   18. The process of embodiments 16-17, wherein oil is recovered during and/or after liquefying the starch-containing material.
   19. The process of any of embodiments 16-18, wherein oil is recovered from the whole stillage.
   20. The process of any of embodiments 16-18, wherein oil is recovered from the thin stillage.
   21. The process of any embodiments 16-18, wherein oil is recovered from the syrup.
   22. The process of any of embodiments 16-21 wherein saccharification and fermentation is performed simultaneously.
   23. The process of any of embodiments 16-22, wherein no nitrogen-compound is present and/or added in steps a)-c), such as during saccharification step b), fermentation step c), or simultaneous saccharification and fermentation (SSF).
   24. The process of any of embodiments 16-22, wherein 10-1,000 ppm, such as 50-800 ppm, such as 100-600 ppm, such as 200-500 ppm nitrogen-compound, preferably urea, is present and/or added in steps a)-c), such as in saccharification step b) or fermentation step c) or in simultaneous saccharification and fermentation (SSF).
   25. The process of any of embodiments 16-24, wherein the alpha-amylase in step a) is from the genus Bacillus, such as a strain of Bacillus stearothermophilus, in particular a variant of a Bacillus stearothermophilus alpha-amylase, such as the one shown in SEQ ID NO: 4.
   26. The process of embodiment 25, wherein the Bacillus stearothermophilus alpha-amylase or variant thereof is truncated, preferably to have around 491 amino acids, such as from 480-495 amino acids.
   27. The process of any of embodiments 25 or 26, wherein the Bacillus stearothermophilus alpha-amylase has a deletion at two positions within the range from positions 179 to 182, such as positions I181+G182, R179+G180, G180+I181, R179+I181, or G180+G182, preferably I181+G182, and optionally a N193F substitution, (using SEQ ID NO: 4 for numbering).
   28. The process of any of embodiments 25-27, wherein the Bacillus stearothermophilus alpha-amylase has a substitution at position S242, preferably S242Q substitution.
   29. The process of any of embodiments 25-28, wherein the Bacillus stearothermophilus alpha-amylase has a substitution at position E188, preferably E188P substitution.
   30. The process of any of embodiments 25-29, wherein the alpha-amylase is selected from the group of Bacillus stearothermophilus alpha-amylase variants with the following mutations in addition to I181*+G182* and optionally N193F:

V59A + Q89R + G112D + E129V + K177L + R179E + K220P + N224L + Q254S;
V59A + Q89R + E129V + K177L + R179E + H208Y + K220P + N224L + Q254S;
V59A + Q89R + E129V + K177L + R179E + K220P + N224L + Q254S + D269E + D281N;
V59A + Q89R + E129V + K177L + R179E + K220P + N224L + Q254S + I270L;
V59A + Q89R + E129V + K177L + R179E + K220P + N224L + Q254S + H274K;
V59A + Q89R + E129V + K177L + R179E + K220P + N224L + Q254S + Y276F;
V59A + E129V + R157Y + K177L + R179E + K220P + N224L + S242Q + Q254S;
V59A + E129V + K177L + R179E + H208Y + K220P + N224L + S242Q + Q254S;
59A + E129V + K177L + R179E + K220P + N224L + S242Q + Q254S;
V59A + E129V + K177L + R179E + K220P + N224L + S242Q + Q254S + H274K;
V59A + E129V + K177L + R179E + K220P + N224L + S242Q + Q254S + Y276F;
V59A + E129V + K177L + R179E + K220P + N224L + S242Q + Q254S + D281N;
V59A + E129V + K177L + R179E + K220P + N224L + S242Q + Q254S + M284T;
V59A + E129V + K177L + R179E + K220P + N224L + S242Q + Q254S + G416V;
V59A + E129V + K177L + R179E + K220P + N224L + Q254S;
V59A + E129V + K177L + R179E + K220P + N224L + Q254S + M284T;
A91L + M96I + E129V + K177L + R179E + K220P + N224L + S242Q + Q254S;
E129V + K177L + R179E;
E129V + K177L + R179E + K220P + N224L + S242Q + Q254S;
E129V + K177L + R179E + K220P + N224L + S242Q + Q254S + Y276F + L427M;
E129V + K177L + R179E + K220P + N224L + S242Q + Q254S + M284T;
El 29V + K177L + R179E + K220P + N224L + S242Q + Q254S + N376* + I377*;
E129V + K177L + R179E + K220P + N224L + Q254S;
E129V + K177L + R179E + K220P + N224L + Q254S + M284T;
E129V + K177L + R179E + S242Q;
E129V + K177L + R179V + K220P + N224L + S242Q + Q254S;
K220P + N224L + S242Q + Q254S;
M284V;
V59A Q89R + E129V + K177L + R179E + Q254S + M284V.

31. The process of any of embodiments 25-30, wherein the alpha-amylase is selected from the group of Bacillus stearothermophilus alpha-amylase variants:

I181*+G182*+N193F+E129V+K177L+R179E;

I181*+G182*+N193F+V59A+Q89R+E129V+K177L+R179E+H208Y+K220P+N224L+Q254S;

I181*+G182*+N193F+V59A Q89R+E129V+K177L+R179E+Q254S+M284V; and

I181*+G182*+N193F+E129V+K177L+R179E+K220P+N224L+S242Q+Q254S (using SEQ ID NO: 4 for numbering).

32. The process of any of embodiments 25-31, wherein the alpha-amylase variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 4.
33. The process of any of embodiments 25-32, wherein the alpha-amylase is present and/or added in a concentration of 0.1-100 micro gram per gram DS, such as 0.5-50 micro gram per gram DS, such as 1-25 micro gram per gram DS, such as 1-10 micro gram per gram DS, such as 2-5 micro gram per gram DS.
34. The process of any of embodiments 16-33, wherein from 1-50 micro gram, particularly from 2-40 micro gram, particularly 4-25 micro gram, particularly 5-20 micro gram Palaeococcus ferrophilus S8A protease per gram DS are present and/or added in liquefaction.
35. The process of any of embodiments 16-34, wherein the Palaeococcus ferrophilus. protease is selected from:
a) a polypeptide comprising or consisting of amino acids 101 to 425 of SEQ ID NO: 2;
b) a polypeptide having at least 80%, at least 85, at least 90%, at least 91%, at least 92%, at least 93%, at least 94%, at least 95%, at least 96%, at least 97%, at least 98%, at least 99%, or 100% sequence identity to amino acids 101 to 425 of SEQ ID NO: 2.
36. The process of any of embodiments 16-35, further wherein the glucoamylase present and/or added in saccharification step b) and/or fermentation step c) is of fungal origin, preferably from a stain of Aspergillus, preferably A. niger, A. awamori, or A. oryzae; or a strain of Trichoderma, preferably T. reesei; or a strain of Talaromyces, preferably T. emersonii, or a strain of Trametes, preferably T. cingulata, or a strain of Pycnoporus, or a strain of Gloeophyllum, such as G. sepiarium or G. trabeum, or a strain of the Nigrofomes.
37. The process of embodiment 36, wherein the glucoamylase is derived from Talaromyces emersonii, such as the one shown in SEQ ID NO: 5 herein.
38. The process of embodiment 37, wherein the glucoamylase is selected from the group consisting of:
(i) a glucoamylase comprising the polypeptide of SEQ ID NO: 5;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 5.
39. The process of embodiments 36, wherein the glucoamylase is derived from Gloeophyllum sepiarium, such as the one shown in SEQ ID NO: 6.
40. The process of embodiments 39, wherein the glucoamylase is selected from the group consisting of:
(i) a glucoamylase comprising the polypeptide of SEQ ID NO: 6;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 6.
41. The process of embodiments 36, wherein the glucoamylase is derived from Gloeophyllum trabeum such as the one shown in SEQ ID NO: 7.
42. The process of embodiment 41, wherein the glucoamylase is selected from the group consisting of: (i) a glucoamylase comprising the polypeptide of SEQ ID NO: 7; (ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 7.
43. The process of any of embodiments 16-42, wherein the glucoamylase is present in saccharification and/or fermentation in combination with an alpha-amylase.
44. The process of embodiment 43, wherein the alpha-amylase is present in saccharification and/or fermentation is of fungal or bacterial origin.
45. The process of embodiment 43 or 44, wherein the alpha-amylase present and/or added in saccharification and/or fermentation is derived from a strain of the genus Rhizomucor, preferably a strain the Rhizomucor pusillus, such as a Rhizomucor pusillus alpha-amylase hybrid having an Aspergillus niger linker and starch-bonding domain, such as the one shown in SEQ ID NO: 8.
46. The process of embodiment 45, wherein the alpha-amylase present in saccharification and/or fermentation is selected from the group consisting of:
(i) an alpha-amylase comprising the polypeptide of SEQ ID NO: 8;
(ii) an alpha-amylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 8.
47. The process of any of embodiments 44-46, wherein the alpha-amylase is derived from a Rhizomucor pusillus with an Aspergillus niger glucoamylase linker and starch-binding domain (SBD), preferably disclosed as SEQ ID NO: 8, preferably having one or more of the following substitutions: G128D, D143N, preferably G128D+D143N (using SEQ ID NO: 8 for numbering).
48. The process of any of embodiments 16-47, further comprising, prior to the liquefaction step a), the steps of:

i) reducing the particle size of the starch-containing material, preferably by dry milling;

ii) forming a slurry comprising the starch-containing material and water.

49. The process of any of embodiments 16-48, wherein at least 50%, preferably at least 70%, more preferably at least 80%, especially at least 90% of the starch-containing material fit through a sieve with # 6 screen.
50. The process of any of embodiments 16-49, wherein the pH in liquefaction is between above 4.5-6.5, such as around 4.8, or a pH between 5.0-6.2, such as 5.0-6.0, such as between 5.0-5.5, such as around 5.2, such as around 5.4, such as around 5.6, such as around 5.8.
51. The process of any of embodiments 16-50, wherein the temperature in liquefaction is above the initial gelatinization temperature, such as in the range from 70-100° C., such as between 75-95° C., such as between 75-90° C., preferably between 80-90° C., especially around 85° C.
52. The process of any of embodiments 16-51, wherein a jet-cooking step is carried out before liquefaction in step a).
53. The process of embodiment 52, wherein the jet-cooking is carried out at a temperature between 110-145° C., preferably 120-140° C., such as 125-135° C., preferably around 130° C. for about 1-15 minutes, preferably for about 3-10 minutes, especially around about 5 minutes.
54. The process of any of embodiments 16-53, wherein saccharification is carried out at a temperature from 20-75° C., preferably from 40-70° C., such as around 60° C., and at a pH between 4 and 5.
55. The process of any of embodiments 16-54, wherein fermentation or simultaneous saccharification and fermentation (SSF) is carried out carried out at a temperature from 25° C. to 40° C., such as from 28° C. to 35° C., such as from 30° C. to 34° C., preferably around about 32° C.
56. The process of any of embodiments 16-55, wherein the fermentation product is recovered after fermentation, such as by distillation.
57. The process of any of embodiments 16-56, wherein the fermentation product is an alcohol, preferably ethanol, especially fuel ethanol, potable ethanol and/or industrial ethanol.
58. The process of any of embodiments 16-57, wherein the starch-containing starting material is whole grains.
59. The process of any of embodiments 16-58, wherein the starch-containing material is derived from corn, wheat, barley, rye, milo, sago, cassava, manioc, tapioca, sorghum, rice or potatoes.
60. The process of any of embodiments 16-59, wherein the fermenting organism is yeast, preferably a strain of Saccharomyces, especially a strain of Saccharomyces cerevisiae.
61. A process according to any of embodiments 16-60, wherein the ratio between alpha-amylase and protease in liquefaction is in the range between 1:1 and 1:50 (micro gram alpha-amylase:micro gram protease), such as between 1:3 and 1:40, such as around 1:4 (micro gram alpha-amylase:micro gram protease).
62. An enzyme composition comprising:
an alpha-amylase, and a Palaeococcus ferrophilus S8A protease, preferably a polypeptide according to embodiments 1-8.
63. The enzyme composition embodiment 62, wherein the ratio between alpha-amylase and protease is in the range from 1:1 and 1:50 (micro gram alpha-amylase:micro gram protease), such as between 1:3 and 1:40, such as around 1:4 (micro gram alpha-amylase:micro gram protease).
64. The enzyme composition of any of embodiments 62-64, wherein the enzyme composition comprises a glucoamylase and the ratio between alpha-amylase and glucoamylase in liquefaction is between 1:1 and 1:10, such as around 1:2 (micro gram alpha-amylase:micro gram glucoamylase).
65. The enzyme composition of any of embodiments 62-64, wherein the alpha-amylase is a bacterial alpha-amylase, particularly derived from Bacillus or Exiguobacterium species, such as, e.g., Bacillus licheniformis or Bacillus stearothermophilus.
66. The enzyme composition of any of embodiments 62-65, wherein the alpha-amylase is from a strain of Bacillus stearothermophilus, in particular a variant of a Bacillus stearothermophilus alpha-amylase, such as the one shown in SEQ ID NO: 4.
67. The enzyme composition of any of embodiments 62-66, wherein the Bacillus stearothermophilus alpha-amylase or variant thereof is truncated, preferably to have around 491 amino acids, such as from 480-495 amino acids.
68. The enzyme composition of any of embodiments 62-67, wherein the Bacillus stearothermophilus alpha-amylase has a deletion at two positions within the range from positions 179 to 182, such as positions I181+G182, R179+G180, G180+I181, R179+I181, or G180+G182, preferably I181+G182, and optionally a N193F substitution, (using SEQ ID NO: 4 for numbering).
69. The enzyme composition of any of embodiments 62-68, wherein the Bacillus stearothermophilus alpha-amylase has a substitution at position S242, preferably S242Q substitution.
70. The enzyme composition of any of embodiments 62-69, wherein the Bacillus stearothermophilus alpha-amylase has a substitution at position E188, preferably E188P substitution.
71. The enzyme composition of any of embodiments 62-70, wherein the alpha-amylase is selected from the group of Bacillus stearothermophilus alpha-amylase variants with the following mutations in addition to deletions I181*+G182* and optionally N193F:

V59A + Q89R + G112D + E129V + K177L + R179E + K220P + N224L + Q254S;
V59A + Q89R + E129V + K177L + R179E + H208Y + K220P + N224L + Q254S;
V59A + Q89R + E129V + K177L + R179E + K220P + N224L + Q254S + D269E + D281N;
V59A + Q89R + E129V + K177L + R179E + K220P + N224L + Q254S + I270L;
V59A + Q89R + E129V + K177L + R179E + K220P + N224L + Q254S + H274K;
V59A + Q89R + E129V + K177L + R179E + K220P + N224L + Q254S + Y276F;
V59A + E129V + R157Y + K177L + R179E + K220P + N224L + S242Q + Q254S;
V59A + E129V + K177L + R179E + H208Y + K220P + N224L + S242Q + Q254S;
59A + E129V + K177L + R179E + K220P + N224L + S242Q + Q254S;
V59A + E129V + K177L + R179E + K220P + N224L + S242Q + Q254S + H274K;
V59A + E129V + K177L + R179E + K220P + N224L + S242Q + Q254S + Y276F;
V59A + E129V + K177L + R179E + K220P + N224L + S242Q + Q254S + D281N;
V59A + E129V + K177L + R179E + K220P + N224L + S242Q + Q254S + M284T;
V59A + E129V + K177L + R179E + K220P + N224L + S242Q + Q254S + G416V;
V59A + E129V + K177L + R179E + K220P + N224L + Q254S;
V59A + E129V + K177L + R179E + K220P + N224L + Q254S + M284T;
A91L + M96I + E129V + K177L + R179E + K220P + N224L + S242Q + Q254S;
E129V + K177L + R179E;
E129V + K177L + R179E + K220P + N224L + S242Q + Q254S;
E129V + K177L + R179E + K220P + N224L + S242Q + Q254S + Y276F + L427M;
E129V + K177L + R179E + K220P + N224L + S242Q + Q254S + M284T;
E129V + K177L + R179E + K220P + N224L + S242Q + Q254S + N376* + I377*;
E129V + K177L + R179E + K220P + N224L + Q254S;
E129V + K177L + R179E + K220P + N224L + Q254S + M284T;
E129V + K177L + R179E + S242Q;
E129V + K177L + R179V + K220P + N224L + S242Q + Q254S;
K220P + N224L + S242Q + Q254S;
M284V;
V59A Q89R + E129V + K177L + R179E + Q254S + M284V.

72. The enzyme composition of any of embodiments 62-71, wherein the alpha-amylase is selected from the group of Bacillus stearomthermphilus alpha-amylase variants with the following mutations:

I181*+G182*+N193F+E129V+K177L+R179E;

I181*+G182*+N193F+V59A+Q89R+E129V+K177L+R179E+H208Y+K220P+N224L+Q254S;

I181*+G182*+N193F+V59A Q89R+E129V+K177L+R179E+Q254S+M284V; and

I181*+G182*+N193F+E129V+K177L+R179E+K220P+N224L+S242Q+Q254S (using SEQ ID NO: 4 for numbering).

73. The enzyme composition of any of embodiments 62-72, wherein the alpha-amylase variant has at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 4.
74. The enzyme composition of any of embodiments 62-73, wherein the Palaeococcus ferrophilus S8A protease has at least 85%, such as at least 90%, such as at least 95%, such as at least 96%, such as at least 97%, such as at least 98%, such as at least 99% identity to amino acids 101 to 425 of SEQ ID NO: 2.
75. The composition of any of embodiments 62-74, comprising a glucoamylase of SEQ ID NO: 11 or a glucoamylase having at least 85%, such as at least 90%, such as at least 95%, such as at least 96%, such as at least 97%, such as at least 98%, such as at least 99% identity SEQ ID NO: 11.
76. The process of any of embodiments 15-61, wherein a glucoamylase of SEQ ID NO: 11 or a glucoamylase having at least 85%, such as at least 90%, such as at least 95%, such as at least 96%, such as at least 97%, such as at least 98%, such as at least 99% identity SEQ ID NO: 11 is present/added during liquefaction.
77. The process according to embodiment 60, wherein the yeast cell expresses a glucoamylase, e.g., the glucoamylase of embodiments 36-42.
78. A use of a Palaeococcus ferrophilus S8A protease in liquefaction of starch-containing material.
79. The use according to embodiment 75, wherein the S8A protease has at least 85%, such as at least 90%, such as at least 95%, such as at least 96%, such as at least 97%, such as at least 98%, such as at least 99% identity to amino acids 101 to 425 of SEQ ID NO: 2.

The present invention is further described by the following examples that should not be construed as limiting the scope of the invention.

### EXAMPLES

### Enzymes and Yeast Used in the Examples

Alpha-Amylase Liquozyme SC: Bacillus stearothermophilus alpha-amylase disclosed herein as

SEQ ID NO: 4, and further having the mutations: I181*+G182*+N193F.

Alpha-Amylase BE369 (AA369): Bacillus stearothermophilus alpha-amylase disclosed herein as SEQ ID NO: 4, and further having the mutations: I181*+G182*+N193F+V59A+Q89R+E129V+K177L+R179E+Q254S+M284V truncated to 491 amino acids (using SEQ ID NO: 4 for numbering).
Glucoamylase Po: Mature part of the Penicillium oxalicum glucoamylase disclosed as SEQ ID NO: 2 in WO 2011/127802 and shown in SEQ ID NO: 11 herein.
Glucoamylase PoAMG498 (GA498): Variant of Penicillium oxalicum glucoamylase having the following mutations: K79V+P2N+P4S+P11F+T65A+Q327F (using SEQ ID NO: 11 for numbering).
Glucoamylase X: Blend comprising Talaromyces emersonii glucoamylase disclosed as SEQ ID NO: 34 in WO99/28448, Trametes cingulata glucoamylase disclosed as SEQ ID NO: 2 in WO 06/69289, and Rhizomucor pusillus alpha-amylase with Aspergillus niger glucoamylase linker and starch binding domain (SBD) disclosed in SEQ ID NO: 8 herein having the following substitutions G128D+D143N using SEQ ID NO: 8 for numbering (activity ratio in AGU:AGU:FAU-F is about 29:8:1).
Yeast: ETHANOL RED™ from Fermentis, USA

### Assays

### Protease Assays

### 1) Kinetic Suc-AAPF-pNA Assay

pNA substrate: Suc-AAPF-pNA (Bachem L-1400).
Temperature: Room temperature (25° C.)
Assay buffers: 100 mM succinic acid, 100 mM HEPES, 100 mM CHES, 100 mM CABS, 1 mM CaCl₂, 150 mM KCI, 0.01% Triton X-100 adjusted to pH-values 2.0, 3.0, 4.0, 5.0, 6.0, 7.0, 8.0, 9.0, 10.0, and 11.0 with HCl or NaOH.
20 μl protease (diluted in 0.01% Triton X-100) was mixed with 100 μl assay buffer. The assay was started by adding 100 μl pNA substrate (50 mg dissolved in 1.0 ml DMSO and further diluted 45× with 0.01% Triton X-100). The increase in OD₄₀₅ was monitored as a measure of the protease activity.

### 2) Endpoint Suc-AAPF-pNA AK Assay

pNA substrate: Suc-AAPF-pNA (Bachem L-1400).
Temperature: controlled (assay temperature).
Assay buffer: 100 mM succinic acid, 100 mM HEPES, 100 mM CHES, 100 mM CABS, 1 mM CaCl₂, 150 mM KCI, 0.01% Triton X-100, pH 7.0.
200 μl pNA substrate (50 mg dissolved in 1.0 ml DMSO and further diluted 45× with the Assay buffer) were pipetted in an Eppendorf tube and placed on ice. 20 μl protease sample (diluted in 0.01% Triton X-100) was added. The assay was initiated by transferring the Eppendorf tube to an Eppendorf thermomixer, which was set to the assay temperature. The tube was incubated for 15 minutes on the Eppendorf thermomixer at its highest shaking rate (1400 rpm.). The incubation was stopped by transferring the tube back to the ice bath and adding 600 μl 500 mM Succinic acid/NaOH, pH 3.5. After mixing the Eppendorf tube by vortexing 200 μl mixture was transferred to a microtiter plate. OD₄₀₅ was read as a measure of protease activity. A buffer blind was included in the assay (instead of enzyme).

### Example 1: Cloning and Expression of S8 Protease from Palaeococcus ferrophilus

Palaeococcus ferrophilus was isolated off the coast of Japan and deposited at DSMZ as DMS No.: 13482 (Takai et al, 2000. International Journal of Systematic and Evolutionary Microbiology, 50, 489-500). A gene encoding a S8 protease was identified on the genome. The gene encoding the S8 protease from Palaeococcus ferrophilus (SEQ ID NO: 1) were codon optimized and synthesized by Gene Art (GENEART AG BioPark, Josef-Engert-Str. 11, 93053, Regensburg, Germany) (synthetic gene: SEQ ID NO: 3). The construct made from the synthetic gene was expressing the gene as an intracellular enzyme without the native secretion signal. . The construct expressing the gene as an intracellular enzyme was made as a linear integration construct where the synthetic gene (without signal) was fused by PCR between two Bacillus subtilis homologous chromosomal regions along with a strong promoter and a chloramphenicol resistance marker. The fusion was made by SOE PCR (Horton, R. M., Hunt, H. D., Ho, S. N., Pullen, J. K. and Pease, L. R. (1989) Engineering hybrid genes without the use of restriction enzymes, gene splicing by overlap extension Gene 77: 61-68). The SOE PCR method is also described in patent application WO 2003095658. In the construct the gene was expressed under the control of a triple promoter system (as described in WO 99/43835), consisting of the promoters from Bacillus licheniformis alpha-amylase gene (amyL), Bacillus amyloliquefaciens alpha-amylase gene (amyQ), and the Bacillus thuringiensis cryIIIA promoter including stabilizing sequence. The plasmid construct and the linear PCR construct where transformed into Bacillus subtilis. Transformants were selected on LB plates supplemented with 6 μg of chloramphenicol per ml. For the construct expressing the intracellular enzyme a recombinant Bacillus subtilis clone was grown in liquid culture. The recombinant enzyme was accumulated in the supernatant upon natural cell lysis. The enzyme containing supernatant was harvested and the enzymes purified as described in Example 2.

### Example 2: Purification and Characterization of S8 Protease from Purification of the S8 Protease from Palaeococcus ferrophilus

The culture broth was centrifuged (20000× g, 20 min) and the supernatant was carefully decanted from the precipitate. The supernatant was filtered through a Nalgene 0.2 μm filtration unit in order to remove the rest of the Bacillus host cells. Solid (NH₄)₂SO₄ was added to the 0.2 μm filtrate to a final concentration of 1.8M (NH₄)₂SO₄ and the enzyme solution was applied to a Butyl Toyopearl column (from Tosoh Haas) equilibrated in 100 mM H₃BO₃, 10 mM MES, 2 mM CaCl₂, 1.8M (NH₄)₂SO₄, pH 6.0. After washing the column extensively with the equilibration buffer, the protease was eluted with a linear gradient between the equilibration buffer and 100 mM H₃BO₃, 10 mM MES, 2 mM CaCl₂, pH 6.0 over four column volumes. Fractions from the column were analysed for protease activity (using the Kinetic Suc-AAPF-pNA assay at pH 9) and the protease activity peak was pooled. The pool from the Butyl Toyopearl column was transferred to 100 mM H₃BO₃, 10mM MES, 2 mM CaCl₂, pH 6.0 on a G25 Sephadex column (from GE Healthcare) and pH of the G25 transferred enzyme was adjusted to pH 9.0 with 3M Tris-base. The pH adjusted solution was applied to a SOURCE 30Q column (from GE Healthcare) equilibrated in 10 mM Tris/HCl, 1 mM CaCl₂, pH 9.0. After washing the column extensively with the equilibration buffer the protease was eluted with a linear gradient over ten column volumes between the equilibration buffer and 10 mM Tris/HCl, 1 mM CaCl₂, 500 mM NaCl, pH 9.0. Fractions from the column were analysed for protease activity (using the Kinetic Suc-AAPF-pNA assay at pH 9) and active fractions were further analysed by SDS-PAGE. Fractions with one dominant band at approx. 37 kDa on the coomassie stained SDS-PAGE gel, were pooled. The pool was the purified preparation and was used for further characterization.

### Characterization of the S8 Protease from Palaeococcus ferrophilus

The kinetic Suc-AAPF-pNA assay was used for obtaining the pH-activity profile and the pH-stability profile for the S8 Protease from Palaeococcus ferrophilus. For the pH-stability profile the protease was diluted 10× in the different Assay buffers to reach the pH-values of these buffers and then incubated for 2 hours at 37° C. After incubation, the pH of the protease incubations was transferred to pH 9.0, before assay for residual activity, by dilution in the pH 9.0 Assay buffer. The endpoint Suc-AAPF-pNA assay was used for obtaining the temperature-activity profile at pH 7.0.
The results are shown in Tables 1-3 below. For Table 1, the activities are relative to the optimal pH for the enzyme. For Table 2, the activities are residual activities relative to a sample, which were kept at stable conditions (5° C., pH 9.0). For Table 3, the activities are relative to the optimal temperature for the enzyme at pH 7.0.

TABLE 1
pH-activity profile

| | S8 Protease from |
|---|---|
| pH | Palaeococcus ferrophilus |
| 2 | 0.00 |
| 3 | 0.00 |
| 4 | 0.01 |
| 5 | 0.02 |
| 6 | 0.22 |
| 7 | 0.66 |
| 8 | 0.98 |
| 9 | 1.00 |
| 10 | 0.86 |
| 11 | 0.56 |

TABLE 2
pH-stability profile
(residual activity after 2 hours at 37° C.)

| | S8 Protease from |
|---|---|
| pH | Palaeococcus ferrophilus |
| 2 | 0.00 |
| 3 | 0.55 |
| 4 | 0.97 |
| 5 | 1.00 |
| 6 | 0.99 |
| 7 | 1.01 |
| 8 | 1.02 |
| 9 | 1.02 |
| 10 | 0.98 |
| 11 | 0.98 |
| After 2 hours | 1.00 |
| at 5° C. | (at pH 9) |

TABLE 3
Temperature activity profile at pH 9.0

| | S8 Protease from |
|---|---|
| Temp (° C.) | Palaeococcus ferrophilus |
| 15 | 0.13 |
| 25 | 0.28 |
| 37 | 0.53 |
| 50 | 0.81 |
| 60 | 0.91 |
| 70 | 1.00 |
| 80 | 0.92 |
| 90 | 0.81 |
| 99 | 0.70 |

### Other Characteristics for the S8 Protease 1 from P. ferrophilus

### Inhibitor: PMSF.

The N-terminal sequence was determined to start at position 101 in SEQ ID NO: 2.
The relative molecular weight as determined by SDS-PAGE was approx. Mᵣ=37 kDa.
The observed molecular weight determined by Intact molecular weight analysis was 33544.3 Da.
The calculated molecular weight from this mature sequence was 33541.8 Da.

### Example 3. Use of the S8 Protease from Palaeococcus ferrophilus in an Ethanol Process

The mature protease of the invention, amino acids 101-425 of SEQ ID NO: 2, was tested for use in a conventional ethanol process on starch slurry including a liquefaction step followed by simultaneous saccharification and fermentation.
Liquefaction: Ten slurries of whole ground corn, thin stillage and tap water were prepared to a total weight of 120 g targeting 32.50% Dry Solids (DS); thin stillage was blended at 30% weight of backset per weight of slurry. Initial slurry pH was approximately 5.2 and was adjusted to 5.0 with either 45% w/v potassium hydroxide or 40% v/v sulfuric acid. A fixed dose of Alpha-Amylase BE369 (2.1 μg EP/gDS) and glucoamylase Po AMG498 (4.5 μg EP/gDS) were applied to all slurries and were combined with S8 protease from Thermococcus litoralis (Tl) (SEQ ID NO: 9), disclosed in WO 2016/196202, or S8 protease from Thermococcus thioreducens (Tt), disclosed herein as SEQ ID NO: 10 and in U.S. provisional application 62/425,655, or S8 protease from P. ferrophilus (Pf) amino acids 101-425 SEQ ID NO: 2 as follows to evaluate the effect of protease treatment during liquefaction:

Control: Alpha-amylase+glucoamylase

Alpha-amylase BE369+glucoamylase PoAMG498+0.5 μg/gDS Tl Protease

Alpha-amylase BE369+glucoamylase PoAMG498+1 μg/gDS Tl Protease

Alpha-amylase BE369+glucoamylase PoAMG498+3 μg/gDS Tl Protease

Alpha-amylase BE369+glucoamylase PoAMG498+0.5 μg/gDS Tt Protease

Alpha-amylase BE369+glucoamylase PoAMG498+1 μg/gDS Tt Protease

Alpha-amylase BE369+glucoamylase PoAMG498+3 μg/gDS Tt Protease

Alpha-amylase BE369+glucoamylase PoAMG498+0.5 μg/gDS Pf Protease

Alpha-amylase BE369+glucoamylase PoAMG498+1 μg/gDS Pf Protease

Alpha-amylase BE369+glucoamylase PoAMG498+3 μg/gDS Pf Protease

Water and enzymes were added to each canister, and then each canister was sealed and mixed well prior to loading into the Labomat. All samples were incubated in the Labomat set to the following conditions: 5° C./min 15 minute ramp to 80° C., hold for 1 min, ramp to 85° C. at 1° C./min and hold for 103 min, 40 rpm for 30 seconds to the left and 30 seconds to the right. Once liquefaction was complete, all canisters were cooled in an ice bath for approximately 20 minutes before proceeding to fermentation.

Simultaneous Saccharification and Fermentation (SSF): Penicillin was added to each mash to a final concentration of 3 ppm and pH was adjusted to 5.0. Next, portions of this mash were transferred to test tubes. All test tubes were drilled with a 1/64″ bit to allow CO, release. Urea was added to half of the tubes to a concentration of 500 ppm. Furthermore, equivalent solids were maintained across all treatments through the addition of water as required to ensure that the urea versus urea-free mashes contained equal solids. Fermentation was initiated through the addition of Glucoamylase X (0.60 AGU/gDS), water and rehydrated yeast. Yeast rehydration took place by mixing 5.5 g of ETHANOL RED™ into 100 mL of 32° C. tap water for at least 15 minutes and dosing 100 μl per test tube.

HPLC analysis: HPLC analysis used an Agilent 1100/1200 combined with a Bio-Rad HPX-87H ion Exclusion column (300 mm×7.8 mm) and a Bio-Rad Cation H guard cartridge. The mobile phase was 0.005 M sulfuric acid and processed samples at a flow rate of 0.6 ml/min, with column and RI detector temperatures of 65 and 55° C., 10 respectively. Fermentation sampling took place after 54 hours by sacrificing 3 tubes per treatment. Each tube was processed by deactivation with 50 μl of 40% v/v H, SO4, vortexing, centrifuging at 1460× g for 10 minutes, and filtering through a 0.45 pm Whatman PP filter. Samples were stored at 4° C. prior to and during HPLC analysis. The method quantified analytes using calibration standards for DP4+, DP3, DP2, glucose, fructose, acetic acid, lactic acid, glycerol and ethanol (% w/v). A four point calibration including the origin is used for quantification.
The obtained ethanol yields are shown in the tables 4 and 5 below.

TABLE 4
Final Ethanol for nitrogen-limited (no urea) fermentations

| | Protease dose | |
|---|---|---|
| Treatment | (μg/gDS) | EtOH(% w/v) |
| BE369 + PoAMG (control) | 0 | 11.272 |
| Control + Tl | 0.5 | 12.0768 |
| Control + Tl | 1 | 12.6484 |
| Control + Tl | 3 | 13.2986 |
| Control + Tt | 0.5 | 12.3314 |
| Control + Tt | 1 | 12.8282 |
| Control + Tt | 3 | 13.4724 |
| Control + Pf | 0.5 | 12.8662 |
| Control + Pf | 1 | 13.3518 |
| Control + Pf | 3 | 13.5792 |

TABLE 5
Final Ethanol for urea based (500 ppm) fermentations

| | Protease dose | |
|---|---|---|
| Treatment | (μg/gDS) | EtOH(% w/v) |
| BE369 + PoAMG (control) | 0 | 13.489 |
| Control + Tl | 0.5 | 13.5632 |
| Control + Tl | 1 | 13.524 |
| Control + Tl | 3 | 13.5262 |
| Control + Tt | 0.5 | 13.6232 |
| Control + Tt | 1 | 13.547 |
| Control + Tt | 3 | 13.5976 |
| Control + Pf | 0.5 | 13.5158 |
| Control + Pf | 1 | 13.6552 |
| Control + Pf | 3 | 13.6518 |

### Example 4: Use of the S8 Protease from Palaeococcus ferrophilus (Pf) for Ethanol Production

The mature protease of the invention, amino acids 101-425 of SEQ ID NO: 2 was tested for use in a conventional ethanol process on starch slurry including a liquefaction step followed by simultaneous saccharification and fermentation.

Liquefaction: Slurries of whole ground corn, thin stillage and tap water were prepared to a total weight of 120 g targeting 32.50% Dry Solids (DS); thin stillage was blended at 30% weight of backset per weight of slurry. Initial slurry pH was approximately 5.2 and was adjusted to 5.0 with either 45% w/v potassium hydroxide or 40% v/v sulfuric acid. A fixed dose of Alpha-Amylase BE369 (2.1 μg EP/gDS) was applied to all slurries and was combined with S8 protease from Thermococcus litoralis (Tl) (SEQ ID NO: 9), disclosed in WO 2016/196202, or S8 protease from Thermococcus thioreducens (Tt), disclosed herein as SEQ ID NO: 10 and in U.S. provisional application 62/425,655, or S8 protease from Palaeococcus ferrophilus (Pf) amino acids 101-425 of SEQ ID NO: 2 as follows to evaluate the effect of protease treatment during liquefaction:

### Control: Alpha-amylase

Alpha-amylase BE369+0.5 μg/gDS Tl Protease
Alpha-amylase BE369+1 μg/gDS Tl Protease
Alpha-amylase BE369+3 μg/gDS Tl Protease

### Alpha-amylase BE369+15 μg/g DS Tl Protease

Alpha-amylase BE369+0.5 μg/gDS Tt Protease
Alpha-amylase BE369+1 μg/gDS Tt Protease
Alpha-amylase BE369+3 μg/gDS Tt Protease

### Alpha-amylase BE369+15 μg/g DS Tt Protease

Alpha-amylase BE369+0.5 μg/gDS Pf Protease
Alpha-amylase BE369+1 μg/gDS Pf Protease
Alpha-amylase BE369+3 μg/gDS Pf Protease
Alpha-amylase BE369+15 μg/gDS Pf Protease
Water and enzymes were added to each canister, and then each canister was sealed and mixed well prior to loading into the Labomat. All samples were incubated in the Labomat set to the following conditions: 5° C./min 15 minute ramp to 80° C., hold for 1 min, ramp to 85° C. at 1° C./min and hold for 103 min, 40 rpm for 30 seconds to the left and 30 seconds to the right. Once liquefaction was complete, all canisters were cooled in an ice bath for approximately 20 minutes before proceeding to fermentation.
Simultaneous Saccharification and Fermentation (SSF): Penicillin was added to each mash to a final concentration of 3 ppm and pH was adjusted to 5.0. Next, portions of this mash were transferred to test tubes. All test tubes were drilled with a 1/64″ bit to allow CO, release. Urea was added to half of the tubes to a concentration of 500 ppm. Furthermore, equivalent solids were maintained across all treatments through the addition of water as required to ensure that the urea versus urea-free mashes contained equal solids. Fermentation was initiated through the addition of Glucoamylase X (0.60 AGU/gDS), water and rehydrated yeast. Yeast rehydration took place by mixing 5.5 g of ETHANOL RED™ into 100 mL of 32° C. tap water for at least 15 minutes and dosing 100 μl per test tube.

HPLC analysis: HPLC analysis used an Agilent 1100/1200 combined with a Bio-Rad HPX-87H ion Exclusion column (300 mm×7.8 mm) and a Bio-Rad Cation H guard cartridge. The mobile phase was 0.005 M sulfuric acid and processed samples at a flow rate of 0.6 ml/min, with column and RI detector temperatures of 65 and 55° C., 10 respectively. Fermentation sampling took place after 54 hours by sacrificing 3 tubes per treatment. Each tube was processed by deactivation with 50 μl of 40% v/v H, SO4, vortexing, centrifuging at 1460× g for 10 minutes, and filtering through a 0.45 pm Whatman PP filter. Samples were stored at 4° C. prior to and during HPLC analysis. The method quantified analytes using calibration standards for DP4+, DP3, DP2, glucose, fructose, acetic acid, lactic acid, glycerol and ethanol (% w/v). A four point calibration including the origin is used for quantification.

The obtained ethanol yields are shown in the tables 6 and 7 below.

TABLE 6
Final Ethanol for nitrogen-limited (no urea) fermentations

| | Protease dose | |
|---|---|---|
| Treatment1 | (μg/gDS) | Ethanol (% w/v) |
| BE369 | 0 | 11.63 |
| BE369 + Tl 0.5 | 0.5 | 12.30 |
| BE369 + Tl 1 | 1 | 12.63 |
| BE369 + Tl 3 | 3 | 13.29 |
| BE369 + Tl 15 | 15 | 13.62 |
| BE369 + Tt 0.5 | 0.5 | 12.70 |
| BE369 + Tt 1 | 1 | 12.91 |
| BE369 + Tt 3 | 3 | 13.46 |
| BE369 + Tt 15 | 15 | 13.59 |
| BE369 + Pf 0.5 | 0.5 | 12.68 |
| BE369 + Pf 1 | 1 | 13.13 |
| BE369 + Pf 3 | 3 | 13.55 |
| BE369 + Pf 15 | 15 | 13.72 |

TABLE 7
Final Ethanol for urea based (500 ppm) fermentations

| | Protease dose | |
|---|---|---|
| Treatment1 | (μg/gDS) | Ethanol (% w/v) |
| BE369 | 0 | 13.41 |
| BE369 + Tl 0.5 | 0.5 | 13.49 |
| BE369 + Tl 1 | 1 | 13.50 |
| BE369 + Tl 3 | 3 | 13.51 |
| BE369 + Tl 15 | 15 | 13.61 |
| BE369 + Tt 0.5 | 0.5 | 13.56 |
| BE369 + Tt 1 | 1 | 13.47 |
| BE369 + Tt 3 | 3 | 13.59 |
| BE369 + Tt 15 | 15 | 13.56 |
| BE369 + Pf 0.5 | 0.5 | 13.53 |
| BE369 + Pf 1 | 1 | 13.56 |
| BE369 + Pf 3 | 3 | 13.58 |
| BE369 + Pf 15 | 15 | 13.68 |

### Example 5: Use of S8 Protease from Palaeococcus ferrophilus (Pf) for Ethanol Production

The mature protease of the invention, amino acids 101-425 of SEQ ID NO: 2, was tested for use in a conventional ethanol process on starch slurry including a liquefaction step followed by simultaneous saccharification and fermentation.
Liquefaction: Slurries of whole ground corn, thin stillage and tap water were prepared to a total weight of 120 g targeting 32.50% Dry Solids (DS); thin stillage was blended at 30% weight of backset per weight of slurry. Initial slurry pH was approximately 5.2 and was adjusted to 5.0 with either 45% w/v potassium hydroxide or 40% v/v sulfuric acid. A fixed dose of Alpha-Amylase BE369 (2.1 μg EP/gDS) was applied to all slurries and was combined with S8 protease from Thermococcus litoralis (Tl) (SEQ ID NO: 9), disclosed in WO 2016/196202, or S8 protease from Thermococcus thioreducens (Tt), disclosed herein as SEQ ID NO: 10 and in U.S. provisional application 62/425,655, or S8 protease from Palaeococcus ferrophilus amino acids 101-425 of SEQ ID NO: 2 as follows to evaluate the effect of protease treatment during liquefaction:

### Control: Alpha-amylase

Alpha-amylase BE369+0.5 μg/gDS Tl Protease
Alpha-amylase BE369+5.0 μg/gDS Tl Protease
Alpha-amylase BE369+0.5 μg/gDS Tf Protease
Alpha-amylase BE369+5.0 μg/gDS Tf Protease
Alpha-amylase BE369+0.5 μg/gDS Pf Protease
Alpha-amylase BE369+5.0 μg/gDS Pf Protease
Water and enzymes were added to each canister, and then each canister was sealed and mixed well prior to loading into the Labomat. All samples were incubated in the Labomat set to the following conditions: 5° C./min 15 minute ramp to 80° C., hold for 1 min, ramp to 85° C. at 1° C./min and hold for 103 min, 40 rpm for 30 seconds to the left and 30 seconds to the right. Once liquefaction was complete, all canisters were cooled in an ice bath for approximately 20 minutes before proceeding to fermentation.
Simultaneous Saccharification and Fermentation (SSF): Penicillin was added to each mash to a final concentration of 3 ppm and pH was adjusted to 5.0. Next, portions of this mash were transferred to test tubes. All test tubes were drilled with a 1/64″ bit to allow CO, release. Urea was added to half of the tubes to a concentration of 500 ppm. Furthermore, equivalent solids were maintained across all treatments through the addition of water as required to ensure that the urea versus urea-free mashes contained equal solids. Fermentation was initiated through the addition of Glucoamylase X (0.60 AGU/gDS), water and rehydrated yeast. Yeast rehydration took place by mixing 5.5 g of ETHANOL RED™ into 100 mL of 32° C. tap water for at least 15 minutes and dosing 100 μl per test tube.
HPLC analysis: HPLC analysis used an Agilent 1100/1200 combined with a Bio-Rad HPX-87H ion Exclusion column (300 mm×7.8 mm) and a Bio-Rad Cation H guard cartridge. The mobile phase was 0.005 M sulfuric acid and processed samples at a flow rate of 0.6 ml/min, with column and RI detector temperatures of 65 and 55° C., 10 respectively. Fermentation sampling took place after 54 hours by sacrificing 3 tubes per treatment. Each tube was processed by deactivation with 50 μl of 40% v/v H, SO4, vortexing, centrifuging at 1460× g for 10 minutes, and filtering through a 0.45 pm Whatman PP filter. Samples were stored at 4° C. prior to and during HPLC analysis. The method quantified analytes using calibration standards for DP4+, DP3, DP2, glucose, fructose, acetic acid, lactic acid, glycerol and ethanol (% w/v). A four point calibration including the origin is used for quantification.
The obtained ethanol yields are shown in the tables below.

TABLE 8
Final Ethanol for nitrogen-limited (no urea) fermentations

| | Protease dose | |
|---|---|---|
| Treatment | (μg/gDS) | Ethanol (% w/v) |
| BE369 | 0 | 11.63 |
| BE369 + Tl 0.5 | 0.5 | 12.30 |
| BE369 + Tl 5 | 5 | 13.50 |
| BE369 + Tt 0.5 | 0.5 | 12.70 |
| BE369 + Tt 5 | 5 | 13.49 |
| BE369 + Pf 0.5 | 0.5 | 12.68 |
| BE369 + Pf 5 | 5 | 13.60 |

TABLE 9
Final Ethanol for urea based (500 ppm) fermentations

| | Protease dose | |
|---|---|---|
| Treatment | (μg/gDS) | Ethanol (% w/v) |
| BE369 | 0 | 13.41 |
| BE369 + Tl 0.5 | 0.5 | 13.49 |
| BE369 + Tl 5 | 5 | 13.52 |
| BE369 + Tt 0.5 | 0.5 | 13.56 |
| BE369 + Tt 5 | 5 | 13.55 |
| BE369 + Pf 0.5 | 0.5 | 13.53 |
| BE369 + Pf 5 | 5 | 13.64 |

### Example 6: Use of S8 Protease from Palaeococcus ferrophilus (Pf) for Ethanol Production

The mature protease of the invention, amino acids 101-425 of SEQ ID NO: 2, was tested for use in a conventional ethanol process on starch slurry including a liquefaction step followed by simultaneous saccharification and fermentation.
Liquefaction: Slurries of whole ground corn, thin stillage and tap water were prepared to a total weight of 120 g targeting 32.50% Dry Solids (DS). Initial slurry pH was approximately 5.8 and was adjusted to 5.0 with 40% v/v sulfuric acid. A fixed dose of Liquozyme SC (0.02% w/w corn) was applied to all slurries and was combined with S8 protease from Thermococcus thioreducens (Tt), disclosed herein as SEQ ID NO: 10 and in U.S. provisional application 62/425,655 or S8 protease from Palaeococcus ferrophilus (Pf) amino acids 101-425 of SEQ ID NO: 2 as follows to evaluate the effect of protease treatment during liquefaction:

### Control: Alpha-amylase Liquozyme SC DS

Alpha-amylase Liquozyme SC+5 μg/gDS Tt Protease
Alpha-amylase Liquozyme SC+5 μg/gDS Pf Protease
Water and enzymes were added to each canister, and then each canister was sealed and mixed well prior to loading into the Labomat. All samples were incubated in the Labomat set to the following conditions: 5° C./min Ramp, 15 minutes Ramp to 80° C., hold for 1 min, Ramp to 85° C. at 1° C./min and holding for 103 min, 40 rpm for 30 seconds to the left and 30 seconds to the right. Once liquefaction was complete, all canisters were cooled in an ice bath for approximately 20 minutes before proceeding to fermentation.
Simultaneous Saccharification and Fermentation (SSF): Penicillin was added to each mash to a final concentration of 3 ppm and pH was adjusted to 5.0. Next, portions of this mash were transferred to test tubes. All test tubes were drilled with a 1/64″ bit to allow CO₂ release. Furthermore, equivalent solids were maintained across all treatments through the addition of water as required to ensure that the mashes contained equal solids. Fermentation was initiated through the addition of Glucoamylase X (0.60 AGU/gDS), water and rehydrated yeast. Yeast rehydration took place by mixing 5.5 g of ETHANOL RED™ into 100 mL of 32° C. tap water for at least 15 minutes and dosing 100 μl per test tube.

HPLC analysis: HPLC analysis used an Agilent 1100/1200 combined with a Bio-Rad HPX-87H ion Exclusion column (300 mm×7.8 mm) and a Bio-Rad Cation H guard cartridge. The mobile phase was 0.005 M sulfuric acid and processed samples at a flow rate of 0.8 ml/min, with column and RI detector temperatures of 65 and 55° C., respectively. Fermentation sampling took place after 54 hours by sacrificing 5 tubes per treatment. Each tube was processed by deactivation with 50 μl of 40% v/v H₂SO₄, vortexing, centrifuging at 1460× g for 10 minutes, and filtering through a 0.2 pm Whatman nylon filter. Samples were stored at 4° C. prior to and during HPLC analysis. The method quantified analytes using calibration standards for DP3, DP2, glucose, fructose, acetic acid, lactic acid, glycerol and ethanol (% w/v). A four-point calibration including the origin is used for quantification.

The obtained ethanol yields are shown in the tables below.

TABLE 10
Final Ethanol for nitrogen-limited (no urea) fermentations

| | Protease dose | |
|---|---|---|
| Treatment | (μg/gDS) | Ethanol (% w/v) |
| Liquozyme SC (control) | 0 | 8.17 |
| Liquozyme SC + Tt | 5 | 12.04 |
| Liquozyme Sc + Pf | 5 | 12.51 |

1. A polypeptide having protease activity, selected from the group consisting of:
(a) a polypeptide having at least 85% sequence identity to the mature polypeptide of SEQ ID NO: 2;
(b) a polypeptide encoded by a polynucleotide that hybridizes under very-high stringency conditions with (i) the mature polypeptide coding sequence of SEQ ID NO: 1, (ii) the full-length complement of (i) or (ii);
(c) a polypeptide encoded by a polynucleotide having at least 85% sequence identity to the mature polypeptide coding sequence of SEQ ID NO: 1;
(d) a fragment of the polypeptide of (a), (b), or (c), that has protease activity.

2. The polypeptide of claim 1, wherein the mature polypeptide is amino acids 101 to 425 of SEQ ID NO: 2.

3. A polynucleotide encoding the polypeptide of claim 1.

4. A nucleic acid construct or recombinant expression vector comprising the polynucleotide of claim 3 operably linked to one or more heterologous control sequences that direct the production of the polypeptide in an expression host.

5. A recombinant host cell comprising the polynucleotide of claim 3 operably linked to one or more heterologous control sequences that direct the production of the polypeptide.

6. A method of producing a polypeptide having protease activity, comprising (a) cultivating the host cell of claim 5 under conditions conducive for production of the polypeptide and (b) optionally recovering the polypeptide.

7. A process for liquefying starch-containing material comprising liquefying the starch-containing material at a temperature above the initial gelatinization temperature in the presence of at least an alpha-amylase and a S8A Palaeococcus ferrophilus protease.

8. A process for producing fermentation products from starch-containing material comprising the steps of:
a) liquefying the starch-containing material at a temperature above the initial gelatinization temperature in the presence of at least:
an alpha-amylase; and
a S8A Palaeococcus ferrophilus protease;
b) saccharifying using a glucoamylase;
c) fermenting using a fermenting organism.

9. A process of recovering oil from a fermentation product production by a process as claimed in claim 8 further comprising the steps of:
d) recovering the fermentation product to form whole stillage;
e) separating the whole stillage into thin stillage and wet cake;
f) optionally concentrating the thin stillage into syrup;
wherein oil is recovered from the:
liquefied starch-containing material after step a) of the process as claimed in claim 8; and/or
downstream from fermentation step c) of the process as claimed in claim 8.

10. The process of claim 8, wherein from 1-50 micro gram Palaeococcus ferrophilus S8A protease per gram DS are present and/or added in liquefaction.

11. The process of claim 8, wherein the Palaeococcus ferrophilus protease is selected from:
a) a polypeptide comprising or consisting of amino acids 101 to 425 of SEQ ID NO: 2; or
b) a polypeptide having at least 80% sequence identity to amino acids 101 to 425 of SEQ ID NO: 2.

12. The process of claim 8, wherein the fermentation product is fuel ethanol.

13. An enzyme composition comprising a Palaeococcus ferrophilus S8A protease according to claim 1.

14. The enzyme composition of claim 13, further comprising an alpha-amylase.

15. (canceled)

16. The polypeptide of claim 1, which has at least 90% sequence identity to the mature polypeptide of SEQ ID NO: 2.

17. The polypeptide of claim 1, which has at least 95% sequence identity to the mature polypeptide of SEQ ID NO: 2.

18. The polypeptide of claim 1, which has at least 97% sequence identity to the mature polypeptide of SEQ ID NO: 2.

19. The polypeptide of claim 1, which has at least 98% sequence identity to the mature polypeptide of SEQ ID NO: 2.

20. The polypeptide of claim 1, which has at least 99% sequence identity to the mature polypeptide of SEQ ID NO: 2.


SEQUENCE LISTING (11 sequences)

SEQ ID NO: 1 - DNA, 1278 bp, Palaeococcus ferrophilus
atgagaaagtacattgcagtggcgctggccatcgcgatgttcgcagggctttttgtggttcccaccatggccgagaggcccgagatggtcagggtcgtggttcacatagacagatccagctttaatcccgcgggggttttgaacctcggggggcacgtcgtgtaccagttccgcctgattgacgccgccgtggttgaggttccctccaccgccgtagggaggctcaagaggctgcccggcgttgagaaagtcgagttcgaccatcaggcagttatcctcaagggcaagccttcgggggctgggaagcccaagccaagccagccaccccaagaggttccatggggaatagagcgcgttaaggctccaagtgtgtggagcacaaccgatggatcaagcaacggggtcatccaggttgcaatcctcgacactggaatagactacgaccacccggacctcgccgccaacctcgcctggggtgtgagcacgcttaggggtagggtttctaccaaaccgaaggactacagagaccagaacggtcacggaacccacgtggcgggaacaatagcggccctcaacaacgacatcggagttgtcggcgtcgctcctggagtccagatttacgcgattagggttctcgacgcgagcggaaggggttcgtacagtgacatagccataggaattgagcaggccatcctcggcccggacggagttgcagacaaggatggagatggaataatagcgggcgatcccgacgatgacgcggcagaggtaataagcatgtccctcggaggctccgccgacgacagctacctccacgatatgataatccaagcctacaacgctggaatagtcatcgtagccgccagcggcaacgagggtgcctcaagcccgagctaccctgccgcttacccggaggttattgccgtcggcgcgagtgacatcaacgacaacatcgcgagcttcagcaaccgccagcctgaggtcagcgccccgggcgttgacgttctcagcacctacccggacgacacttacaagactctcagcggaacgagcatggccacacctcacgtgagcggcgtcgtggcactaatacaagcggctcacttcaacaagtacggcacgatccttccggttggaacctttgacgacatgagcaagaacaccgtgaggggaatcctccacatcacggctgacgaccttggaagcccaggatgggatgtagactacggctacggtattgtgagggcagacctcgccgtccaggccgcgctcggatga

SEQ ID NO: 2 - protein, 425 aa, Palaeococcus ferrophilus
MRKYIAVALAIAMFAGLFVVPTMAERPEMVRVVVHIDRSSFNPAGVLNLGGHVVYQFRLIDAAVVEVPSTAVGRLKRLPGVEKVEFDHQAVILKGKPSGAGKPKPSQPPQEVPWGIERVKAPSVWSTTDGSSNGVIQVAILDTGIDYDHPDLAANLAWGVSTLRGRVSTKPKDYRDQNGHGTHVAGTIAALNNDIGVVGVAPGVQIYAIRVLDASGRGSYSDIAIGIEQAILGPDGVADKDGDGIIAGDPDDDAAEVISMSLGGSADDSYLHDMIIQAYNAGIVIVAASGNEGASSPSYPAAYPEVIAVGASDINDNIASFSNRQPEVSAPGVDVLSTYPDDTYKTLSGTSMATPHVSGVVALIQAAHFNKYGTILPVGTFDDMSKNTVRGILHITADDLGSPGWDVDYGYGIVRADLAVQAALG

SEQ ID NO: 3 - DNA, 1209 bp, Artificial
  Codon optimized sequence
atggaaagaccggaaatggttagagttgttgtccatattgatcgctcatcatttaatccggcaggcgttctgaatctgggaggccatgttgtttatcagtttagactgattgatgcagcagttgttgaagttccgtcaacagcagttggcagactgaaaagactgcctggcgttgaaaaagtcgaatttgatcatcaagcggtcatcctgaaaggcaaaccgtcaggcgcaggcaaaccgaaaccgtcacaaccgcctcaagaagttccttggggcattgaaagagttaaagcaccgtcagtttggtcaacaacggatggctcatcaaatggcgttattcaagttgcaattctggatacaggcatcgactatgatcatccggatctggcagcaaatctggcatggggagtttcaacactgagaggcagagtttcaacaaaaccgaaagattatagagatcagaatggccatggcacacatgttgcaggcacaattgcagcactgaataatgatattggcgttgttggagttgcacctggcgttcaaatttatgcaattagagttctggatgcatcaggcagaggctcatattcagatattgcaattggcattgaacaggcaattctgggaccggatggcgttgcagataaagatggcgacggcattattgcaggcgatcctgatgatgatgcagcggaagttatttcaatgtcactgggaggctcagcagatgattcatatcttcatgatatgatcatccaggcgtataatgcaggcattgttattgttgcagcatcaggcaatgaaggcgcatcatcaccgtcatatccggcagcatatccggaagttattgcagttggcgcatcagatattaacgataacattgcgagctttagcaatagacaaccggaagtttcagcacctggcgtcgatgttctgtcaacatatccggatgatacgtataaaacactgtcaggcacatcaatggcaacaccgcatgtttcaggcgttgttgcactgattcaagcagcacattttaacaaatatggcacaattctgccggtcggcacatttgatgatatgagcaaaaatacagtccgtggcattctgcatattacagcagatgatctgggctcaccgggatgggatgttgattatggctatggcattgttagagcagatctggcagttcaagcagcgctgggctaa

SEQ ID NO: 4 - protein, 515 aa, Bacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 5 - protein, 591 aa, Talaromyces emersonii
ATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 6 - protein, 556 aa, Gloeophyllum sepiarium
QSVDSYVSSEGPIAKAGVLANIGPNGSKASGASAGVVVASPSTSDPDYWYTWTRDSSLVFKSLIDQYTTGIDSTSSLRTLIDDFVTAEANLQQVSNPSGTLTTGGLGEPKFNVDETAFTGAWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSVYSINSGVASNAAVATGRYPEDSYQGGNPWYLTTFAVAEQLYDALNVWESQGSLEVTSTSLAFFQQFSSGVTAGTYSSSSSTYSTLTSAIKNFADGFVAINAKYTPSNGGLAEQYSKSDGSPLSAVDLTWSYASALTAFEARNNTQFAGWGAAGLTVPSSCSGNSGGPTVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 7 - protein, 559 aa, Gloeophyllum trabeum
QSVDSYVGSEGPIAKAGVLANIGPNGSKASGAAAGVVVASPSKSDPDYWYTWTRDSSLVFKSLIDQYTTGIDSTSSLRSLIDSFVIAEANIQQVSNPSGTLTTGGLGEPKFNVDETAFTGAWGRPQRDGPALRATALITYGNWLLSNGNTTWVTSTLWPIIQNDLNYVVQYWNQTTFDLWEEVNSSSFFTTAVQHRALREGAAFATKIGQTSSVSSYTTQAANLLCFLQSYWNPTSGYITANTGGGRSGKDANTLLASIHTYDPSAGCDATTFQPCSDKALSNLKVYVDSFRSVYSINSGIASNAAVATGRYPEDSYQGGNPWYLTTFAVAEQLYDALNVWAAQGSLNVTSISLPFFQQFSSSVTAGTYASSSTTYTTLTSAIKSFADGFVAINAQYTPSNGGLAEQFSRSNGAPVSAVDLTWSYASALTAFEARNNTQFAGWGAVGLTVPTSCSSNSGGGGGSTVAVTFNVNAQTVWGENIYITGSVDALSNWSPDNALLLSSANYPTWSITVNLPASTAIQYKYIRKNNGAVTWESDPNNSITTPASGSVTENDTWR

SEQ ID NO: 8 - protein, 583 aa, Artificial
  Hybrid alpha-amylase comprising catalytic domain from Rhizomucor pusillus alpha-amylase fused to linker and starch binding domain from Aspergillus niger glucoamylase
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 9 - protein, 424 aa, Thermococcus litoralis
MEFNKVFSLLLVFVVLGATAGIVGAVSAEKVRVIITIDKDFNENSVFALGGNVVARGKVFPIVIAELSPRAVERLKNAKGVVRVEYDAEVQVLKGKSPGAGKPKPSQPAQTIPWGIERIKAPDVWSITDGSSSGVIEVAILDTGIDYDHPDLAANLAWGVSVLRGKVSTKPKDYKDQNGHGTHVAGTVAALNNDIGVVGVAPAVEIYAVRVLDASGRGSYSDIILGIEQALLGPDGVLDSDGDGIIVGDPDDDAAEVISMSLGGLSDVQAFHDAIIEAYNYGVVIVAASGNEGASSPSYPAAYPEVIAVGATDVNDQVPWWSNRGVEVSAPGVDVLSTYPDDSYETLSGTSMATPHVSGVVALIQAAYYNKYGSVLPVGTFDDNTMSTVRGILHITADDLGSSGWDADYGYGIVRADLAVQAVN

SEQ ID NO: 10 - protein, 422 aa, Thermococcus thioreducens
MGRKDITIALVALIVLSLLGVPATAEKPELVRVIVHVDRGHFNTADVATIGGHVVYQFKLIDAVVVEVPSTAVGRLKKLPGVKMVEFDHKARILAGPPSWLGGGQPSQQIPWGISRVRAPDVWGITDGSGGVIEVAVLDTGVDYDHPDLAGNIAWCVSTLRGRVTTNPAQCKDQNGHGTHVIGTIAALNNDIGVVGVAPGVEIYSIRVLDASGSGSYSDIAIGIEQALLGPDGILDKDGDGIIVGDPDDDAAEVISMSLGGPTDDQYLHDMIITAYNYGVVIVAASGNEGASSPSYPAAYPEVIAVGASDVNDQIASWSNRQPEVSAPGVDILSTYPDDTYETLSGTSMATPHVSGVVALIQAAYYNKYGKVLPVGTFDDMGTNTVRGILHVTADDLGDAGWDIYYGYGIVRADLAVQAAIG

SEQ ID NO: 11 - protein, 595 aa, Penicillium oxalicum
RPDPKGGNLTPFIHKEGERSLQGILDNLGGRGKKTPGTAAGLFIASPNTENPNYYYTWTRDSALTAKCLIDLFEDSRAKFPIDRKYLETGIRDYKSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF